(19) United States
(12) Patent Application Publication
(10) Pub. No.: US 2024/0043396 A1
(43) Pub. Date: Feb. 8, 2024

(54) METHODS OF TREATING OCULAR FIBROTIC PATHOLOGIES

(71) Applicant: Mayo Foundation for Medical Education and Research, Rochester, MN (US)

(72) Inventors: Andrew J. Haak, Rochester, MN (US); Daniel J. Tschumperlin, Rochester, MN (US); Sophie Bakri, Rochester, MN (US)

(21) Appl. No.: 18/265,472

(22) PCT Filed: Jan. 27, 2022
(86) PCT No.: PCT/US2022/014045

§ 371 Date: Jun. 6, 2023

Related U.S. Application Data
(60) Provisional application No. 63/143,525, filed on Jan. 29, 2021.

(51) Int. Cl. C07D 311/04; C07D 405/04; C07D 407/04; C07D 221/08; C07D 491/052; C07D 401/10; C07D 405/10; C07D 491/16; C07D 471/04

(52) U.S. Cl. CPC C07D 311/04; C07D 405/04; C07D 407/04; C07D 221/08; C07D 491/052; C07D 401/10; C07D 405/10; C07D 491/16; C07D 471/04

(57) ABSTRACT

The present application provides compounds and methods for treating ocular fibrotic pathologies, including using D1 and/or D5 receptor agonists for treating proliferative vitreoretinopathy.

### CLAIM OF PRIORITY

This application claims priority to U.S. Patent Application Ser. No. 63/143,525, filed on Jan. 29, 2021, the entire contents of which are hereby incorporated by reference.

### TECHNICAL FIELD

This invention relates to compounds and methods useful in treating ocular fibrotic pathologies, such as proliferative vitreoretinopathy, diabetic retinopathy, and age-related macular degeneration and many others.

### BACKGROUND

Fibrosis affects many parts of the eye and is a pathway in many eye diseases, and also can be an unwanted complication of treatment. Fibrosis occurs in diabetic retinopathy, epiretinal membranes, proliferative vitreoretinopathy, macular degeneration, choroidal neovascularization and any eye diseases that result from angiogenesis. After surgery for glaucoma (e.g. trabeculectomy and filtering procedures), fibrosis can result in surgical failure and need for an anti-fibrotic.

### SUMMARY

The present disclosure provides various methods of using dopamine receptor agonists, including compounds of Formulae (I)-(IV) disclosed herein. These methods include inhibiting epithelial to mesenchymal transition (EMT), inhibiting migration or proliferation, inhibiting expression of a profibrotic gene, inhibiting extra-cellular matrix production and deposition, and enhancing extra-cellular matrix degradation in a retinol pigment epithelial (RPE) cell, including in vitro, in vivo, and ex vivo. The methods also include treatment and prevention of fibrosis in ocular tissues, including treating or preventing an ocular fibrotic pathology, such as proliferative vitreoretinopathy. Certain embodiments or these methods and compounds are described herein.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 contains a bar graph showing expression of all GPCR known to exclusively couple to G alpha S to promote antifibrotic signaling. Expression values are extracted from publically available datasets previously published (GSE12548). DRD5 is highlighted

FIG. 2A contains images showing epithelial to mesenchymal transition (EMT) in cultured RPE cells. ARPE-19 cells were cultured +/−5 ng/mL TGFβ1 for 24 hours in EMEM containing 0% FBS along with the indicated concentration of CTC-6 or MS-9, dopamine receptor agonists. Phase contrast images are depicted to depict changes in cellular morphology.

FIG. 2B contains bar graphs showing epithelial to mesenchymal transition (EMT) in cultured RPE cells. ARPE-19 cells were cultured +/−5 ng/mL TGFβ1 for 24 hours in EMEM containing 0% FBS along with the indicated concentration of CTC-6 or MS-9, dopamine receptor agonists. RNA expression is shown relative to the control treated cells, GAPDH.

FIG. 3A contains a bar graph showing dopamine receptor expression and dopamine synthesis in culture RPE cells. Expression of dopamine receptor family in cultured ARPE-19 cells treated ±TGFβ for 24 hours. Results are expressed as the mean±s.e.m. Y-axis is logarithmic.

FIG. 3B contains a bar graph showing in vitro dopamine production by cultured ARPE-19 cells treated ±TGFβ for 24 hours. n=4 independent experiments. Comparison made by paired t-test, * p<0.05. Results are expressed as the mean±s.e.m.

FIG. 3C contains a schematic diagram showing D5 dopamine receptor activation promote antifibrotic gene expression. Coupling of D5 dopamine receptor to Gas is activated by fenoldopam (FNP), a D5 dopamine receptor agonist, and inhibited by SCH 39166 (SCH), a D5 dopamine receptor antagonist.

FIG. 3D contains a bar graph showing effect of agonists and antagonists of the D5 dopamine receptor on profibrotic gene expression in cultured ARPE-19 cells. ARPE-19 cells were treated ±TGFβ, Fenoldopam (FNP), and SCH 39166 (SCH) for 24 hours. n=3 independent experiments. Comparison made by ANOVA, * p<0.05, ** p<0.01, *** p<0.001.

FIG. 4A shows antifibrotic effect of D5 receptor agonist fenoldopam. The figure shows wound migration assay. ARPE-19 cells were plated to confluence prior to the formation of a “wound”. Cells were then treated ±TGFβ, ±1011M fenoldopam and the wound area was imaged and quantified every 24 hours and media changed every 24 hours. Show are representative images from the 0- and 72-hour timepoints for each of the conditions. n=3 independent experiments.

FIG. 4B contains bar graphs showing cellular proliferation of ARPE-19 cells treated ±2% FBS, ±1011M fenoldopam for 4 days. Proliferation was assessed by Cell-titer Fluor. n=3 independent experiments. Comparison made by ANOVA, * p<0.05, *** p<0.001. Results are expressed as the mean±s.e.m.

FIG. 4C shows Live/Dead assessment of ARPE-19 cells treated for 4 days ±2% FBS, ±10 μM fenoldopam. Data are expressed as the number of individual cells within the field of view identified as Live (green), or dead (red). Show are representative images after 4 days in culture for each of the condition n=3 independent experiments. Comparison made by ANOVA, * p<0.05, *** p<0.001. Results are expressed as the mean±s.e.m.

FIG. 4D shows fibronectin deposition of ARPE-19 cells cultured for 4 days ±TGFβ, ±10 μM fenoldopam. Cells were fixed and immunostained for fibronectin and counterstained with DAPI. Show are representative images for each of the condition. Fibronectin intensity, relative to DAPI cell counts was quantified using automated imaging software (Gens). n=3 independent experiments. Comparison made by ANOVA, *** p<0.001, **** p<0.0001. Results are expressed as the mean±s.e.m.

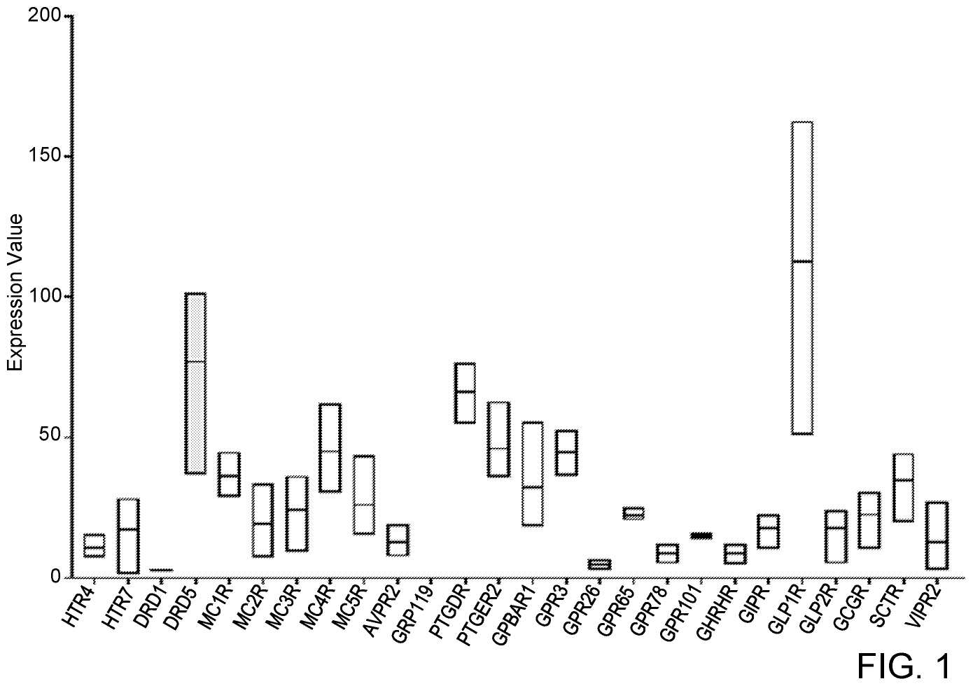

### DETAILED DESCRIPTION

Retinol pigment epithelial (RPE) cells play an important role in maintaining the structural and functional health of the retinal, macular, and associated vasculature. RPE cells form a monocellular layer immediately behind the retina and play an essential role in light absorption, barrier function, and fluid/ion transport. Dysfunction of these cells plays a role in multiple ocular diseases including age-related macular degeneration and proliferative vitreoretinopathy. Aging, inflammation, and acute injury (e.g., retinal injury due to retinal detachment) can all lead to epithelial to mesenchymal transition (EMT) in RPE cells (trans-differentiation into fibroblast-like to mesenchymal cells), stimulating cellular proliferation, migration, and deposition of extracellular matrix (ECM, e.g., type I collagen and fibronectin); all of which contribute to ocular fibrosis and lead to associated diseases (e.g., PVR).

Accordingly, in some embodiments, the present disclosure provides a method of inhibiting epithelial to mesenchymal transition (EMT) in a retinol pigment epithelial (RPE) cell, the method comprising contacting the cell with an effective amount of a compound as disclosed herein, or a pharmaceutically acceptable salt thereof. In some embodiments, the contacting is carried out in vitro, in vivo, or ex vivo. Hence, in some embodiments, the present disclosure provides a method of inhibiting epithelial to mesenchymal transition (EMT) in a retinol pigment epithelial (RPE) cell of a subject, the method comprising administering to the subject a therapeutically effective amount of a compound as disclosed herein, or a pharmaceutically acceptable salt thereof.

Without being bound by any theory, it is believed that activation (agonism) of a G protein coupled receptor (“GPCR”) in RPE cells blocks (or inhibits) EMT in these cells (e.g., agonizing GPCR blocks expression of genes associated with EMT). Hence, agonizing a GPCR in RPE cells allows to maintain epithelial nature of these cells and to maintain RPE cell function.

Accordingly, in some embodiments, the present disclosure provides a method of agonizing a G protein coupled receptor in a retinol pigment epithelial (RPE) cell, the method comprising contacting the cell with an effective amount of a compound as disclosed herein, or a pharmaceutically acceptable salt thereof. In some embodiments, the contacting is carried out in vitro, in vivo, or ex vivo. Hence, in some embodiments, the present disclosure provides a method of agonizing a G protein coupled receptor in a retinol pigment epithelial (RPE) cell of a subject, the method comprising administering to the subject a therapeutically effective amount of a compound as disclosed herein, or a pharmaceutically acceptable salt thereof.

G protein coupled receptors are linked to effector proteins from four main classes of G-proteins (e.g., Gα_{12/13}, Gα_{q/11}, Gα_{i/o}, or Gαₛ). Without being bound by any theory, it is believed that exclusively (or selectively) agonizing Gas-protein coupled receptor in a retinol pigment epithelial (RPE) cell contributes to inhibition of EMT and maintaining epithelial nature in these cells. Accordingly, in some embodiments, the present disclosure provides a method of agonizing Gas-protein coupled receptor in a retinol pigment epithelial (RPE) cell. In some embodiments, this agonizing is selective, e.g., the agonizing is 100-fold, 50-fold, or 10-fold selective to Gas protein coupled receptor as compared to Gα_{12/13}, Gα_{q/11} or Gα_{i/o} protein coupled receptor, or any combination of the aforementioned).

Without being bound by any theory, it is believed that dopamine receptors in a retinol pigment epithelial (RPE) cell (e.g., any of D1, D2, D3, D4, or D5 dopamine receptor) couple exclusively to the Gαₛ protein. Accordingly, in some embodiments, the present disclosure provides a method of agonizing a dopamine receptor in a retinol pigment epithelial (RPE) cell (e.g., D1, D2, D3, D4, or D5 dopamine receptor), the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method of agonizing dopamine receptor in a retinol pigment epithelial (RPE) cell of a subject, the method comprising administering to the subject a therapeutically effective amount of a compound disclosed herein, or a pharmaceutically acceptable salt thereof.

Without being bound by any particular theory or speculation, it is believed that selective D1 and D5 receptor agonism inhibits fibrosis (e.g., lung and cardiac), consistent with known coupling of those receptors to Gαₛ, elevation of cAMP, and inhibition of downstream transcriptional programs such as YAP/TAZ and MRTFA/B.

Accordingly, in some embodiments, the present disclosure provides a method of agonizing dopamine receptor D1 (DRD1) in a retinol pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method of agonizing dopamine receptor D1 (DRD1) in a retinol pigment epithelial (RPE) cell of a subject, the method comprising administering to the subject a therapeutically effective amount of a compound disclosed herein, or a pharmaceutically acceptable salt thereof. In some embodiments, agonizing DRD1 is selective with respect to DRD1 (e.g., the method does not substantially involve agonizing D2, D3, D4, or D5 receptor, or any combination of the aforementioned). For example, the agonizing is 100-fold, 50-fold, or 10-fold selective to D1 dopamine receptor. In some embodiments, DRD1 is preferentially expressed in a retinol pigment epithelial (RPE) cell. For example, DRD1 comprises 51%, 60%, 80%, 90%, 95%, 99%, or 100% of all dopamine receptors expressed in the RPE cell.

In some embodiments, the present disclosure provides a method of agonizing dopamine receptor D5 (DRD5) in a retinol pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method of agonizing dopamine receptor D5 (DRD5) in a retinol pigment epithelial (RPE) cell of a subject, the method comprising administering to the subject a therapeutically effective amount of a compound disclosed herein, or a pharmaceutically acceptable salt thereof. In some embodiments, agonizing DRD5 is selective with respect to DRD5 (e.g., the method does not substantially involve agonizing D1, D2, D3, or D4 receptor, or any combination of the aforementioned). For example, the agonizing is 100-fold, 50-fold, or 10-fold selective to D5 dopamine receptor. For example, DRD5 comprises 51%, 60%, 80%, 90%, 95%, 99%, or 100% of all dopamine receptors expressed in the RPE cell.

In some embodiments, the present disclosure provides a method of agonizing dopamine receptor D1 (DRD1) and dopamine receptor D5 (DRD5) in a retinol pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method of agonizing dopamine receptor D1 (DRD1) and dopamine receptor D5 (DRD5) in a retinol pigment epithelial (RPE) cell of a subject, the method comprising administering to the subject a therapeutically effective amount of a compound disclosed herein, or a pharmaceutically acceptable salt thereof. In some embodiments, agonizing DRD1 and DRD5 is selective with respect to these receptors (e.g., the method does not substantially involve agonizing D2, D3, or D4 receptor, or any combination of the aforementioned). For example, the agonizing is 100-fold, 50-fold, or 10-fold selective to D1 and D5 dopamine receptors. In some embodiments, the method comprises indiscriminately agonizing D1 and D5. In some embodiments, the RPE cell comprises 100×, 50×, or 20× greater amount of D1 receptors compared to D5 receptors. In some embodiments, the RPE cell comprises 200×, 100×, 50×, or 20× greater amount of D5 receptors compared to D1 receptors. In some embodiments, DRD5 is the only type of dopamine receptor expressed in the RPE cell.

Without being bound by any theory, it is believed that agonizing G protein coupled receptors in RPE cells, such as those that couple exclusively to G alpha S (Gₛ) and/or the dopamine receptors described above, inhibits and/or stops and/or prevents these cells from proliferation, migration, and/or secretion of components of extracellular matrix. While inactivation (inhibition or antagonism) of GPCR results in expression of profibrotic genes, such as Acta2 (αSMA), Ctgf (Connective tissue growth factor), Fn1 (Fibronectin), Colla1 (Collagen I), Colla2 (Collagen II), and Col3a1 (Collagen III); activation (or agonism) of GPCR inhibits expression of these genes and therefore prevents fibrotic scarring in ocular tissues.

Accordingly, in some embodiments, the present disclosure provides a method of inhibiting expression of a profibrotic gene in a retinol pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method inhibiting expression of a profibrotic gene in a retinol pigment epithelial (RPE) cell of a subject, the method comprising administering to the subject a therapeutically effective amount of a compound disclosed herein, or a pharmaceutically acceptable salt thereof. In some embodiments, the profibrotic gene is selected from Acta2 (α-smooth muscle actin, αSMA), Ctgf (Connective tissue growth factor), Fn1 (Fibronectin), Colla1 (Collagen I), Colla2 (Collagen II), and Col3a1 (Collagen III), or any combination thereof.

In some embodiments, the present disclosure provides a method of inhibiting proliferation of a retinol pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method inhibiting proliferation of a retinol pigment epithelial (RPE) cell in a subject, the method comprising administering to the subject a therapeutically effective amount of a compound disclosed herein, or a pharmaceutically acceptable salt thereof.

In some embodiments, the present disclosure provides a method of inhibiting migration of a retinol pigment epithelial (RPE) cell (e.g., in an ocular tissue), the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method inhibiting migration of a retinol pigment epithelial (RPE) cell (e.g., in an ocular tissue) in a subject, the method comprising administering to the subject a therapeutically effective amount of a compound disclosed herein, or a pharmaceutically acceptable salt thereof.

In some embodiments, the present disclosure provides a method of inhibiting secretion of a component of extracellular matrix from a retinol pigment epithelial (RPE) cell, the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method of inhibiting secretion of a component of extracellular matrix from a retinol pigment epithelial (RPE) cell of a subject, the method comprising administering to the subject a therapeutically effective amount of a compound disclosed herein, or a pharmaceutically acceptable salt thereof. The method includes inhibiting extra-cellular matrix production and deposition by an RPE cell.

Suitable examples of components of extracellular matrix include proteins, glycosaminoglycans (mucopolysaccharides), and glycoconjugates (glycans, or polysaccharides, that are covalently linked to proteins, peptides, or lipids). Examples of glycoconjugates of the extracellular matrix include glycoproteins, proteoglycans, glycopeptides, peptidoglycans, glycolipids, glycosides, and lipopolysaccharides, or any combination of the aforementioned. Examples of glycosaminoglycans of the extracellular matrix include hyaluronic acid, chondroitin sulfate, dermatan sulfate, keratan sulfate, and heparan sulfate. Examples of proteoglycans of the extracellular matrix include aggrecan, versican, neurocan, and brevican. Examples of glycoproteins include tenascin, fibronectin, laminin, osteopontin, fibulin, and matricellar glycoproteins, or any combination of the aforementioned. Examples of proteins of the extracellular matrix include collagen (type I, II, III, IV, V, or VI), elastin, tropoeslastin, fibrillin, fibrin, fibrinogen, fibronectin, and laminin, or any combination of the aforementioned.

In some embodiments, the present disclosure provides a method of inhibiting deposition and/or accumulation of extracellular matrix in an ocular tissue (e.g., in or near an RPE cell), the method comprising contacting the cell with an effective amount of a compound disclosed herein, or a pharmaceutically acceptable salt thereof. In some embodiments, the contacting is carried out in vitro, in vivo, or ex vivo. Hence, in some embodiments, the present disclosure provides a method of inhibiting deposition and/or accumulation of extracellular matrix in an ocular tissue (e.g., in or near an RPE cell) of a subject, the method comprising administering to the subject a therapeutically effective amount of a compound disclosed herein, or a pharmaceutically acceptable salt thereof. The method includes enhancing extra-cellular matrix degradation by an RPE cell.

In some embodiments, the present disclosure provides a method of reversing fiber formation and extracellular matrix accumulation in an ocular tissue. Without being bound by any theory, it is believed that agonizing GPCR and/or dopamine receptor in an RPE cell (e.g., as described herein) reverses formation of components of extracellular matrix in an ocular tissue and results in dissolution of the extracellular matrix that already accumulated in the ocular tissue.

In some embodiments, epithelial to mesenchymal transition (EMT) in RPE cells, stimulating RPE cellular proliferation and/or migration, and/or extracellular matrix (ECM) deposition (fibrosis, scarring) in an ocular tissue is induced by (or results from) trauma, recovery after surgery (e.g., cataract surgery), ocular tissue injury (e.g., open globe injury), aging, inflammation, infection (e.g., bacterial, fungal, or viral infection), intraocular pressure, genetic predisposition, co-morbidity, damage to optic nerve, tissue ischemia, retinal detachment, vascular leakage, hemorrhage, or any combination of these factors. Normally cells in the ocular tissues, such as the RPE cells, generate just the right amount of tissue to replace old tissue or repair damage. Excessive connective tissue generation (e.g., in response to trauma or injury as discussed above) results in pathological accumulation of fibrotic tissue (e.g., extracellular matrix proteins) leading to tissue thickening, fibrosis, and scarring.

In some embodiments, the present disclosure provides a method of inhibiting (or reversing) an ocular tissue fibrosis (inhibiting fibrosis in an ocular tissue). Suitable examples of ocular tissues include iris, cornea, retina (including neural retina), retinol pigment epithelium, choriocapillaris, sclera, nerve fibers, ganglion cells, choroid, choroidal vessels, uvea, ciliary body, forvea, Schlemm's canal, corneal stroma, and macula. In some embodiments, the ocular tissue fibrosis is associated with RPE cells.

In some embodiments, the present disclosure provides a method of treating or preventing an ocular fibrotic pathology (e.g., an ocular disease or condition in which fibrosis is implicated) in a subject, the method comprising administering to the subject in need thereof a therapeutically effective amount of any one of the compounds described herein, or a pharmaceutically acceptable salt thereof.

In some embodiments, the subject in need of treatment of an ocular fibrotic pathology is diagnosed with an ocular fibrotic pathology by a treating physician. In some embodiments, the subject in need of prevention of an ocular fibrotic pathology is diagnosed with an ocular tissue trauma or injury, ocular infection, increased intraocular pressure, retinal or subretinal neovascularization, genetic predisposition, co-morbidity (e.g., diabetes or another metabolic disease), damage to optic nerve, or a similar condition, by a treating physician. Suitable examples of ocular tissue injuries include a drug-induced injury (injury caused by an antibiotic or an anticancer drug), tissue injury caused by autoimmune disease, including sepsis, tissue ischemia, vascular leakage, hemorrhage, including subretinal hemorrhage, macula edema, chronic wound healing, and injury caused by an infection.

Ocular fibrosis contributes to visual loss in millions of people globally. The compounds within the present claims (e.g., D1 and/or D5 receptor agonists) advantageously treat or prevent the ocular fibrotic pathologies and therefore prevent or decrease the visual loss.

In some embodiments, an ocular fibrotic pathology is selected from: proliferative vitreoretinopathy (“PVR”), epiretinal membrane, diabetic retinopathy, ischemic retinopathy, macular degeneration, age-related macular degeneration (“ARMD,” including dry ARMD and neovascular ARMD), keratitis, pterygia, pingueculae, retinopathy of prematurity, glaucoma (including neovascular glaucoma, open-angle glaucoma, angle-closure glaucoma, secondary glaucoma, and childhood glaucoma), Stargardt's disease, sickle cell retinopathy, radiation retinopathy, optic neuropathy, retinal detachment, retinal degeneration, uveitis, dry eye disease, congenital fibrosis of the extraocular muscles (“CFEOM”), and corneal fibrosis. In some embodiments, the ocular fibrotic pathology is selected from: opacification and fibrosis of the posterior capsule of the lens following eye surgery, fibrosis following glaucoma filtration surgery, fibrosis following a wound or trauma, conjunctival fibrosis or subconjunctival fibrosis, fibrosis of the ocular muscles, Graves disease, fibrosis following wound healing of the skin around the eye and face, fibrosis of the surface of the eye with pterygium or pingueculae, fibrosis due to choroidal neovascularization and angiogenesis, fibrosis following a corneal wound, fibrosis following corneal laser surgery, fibrosis following refractive surgery, and fibrosis following a corneal transplant.

Common symptoms of the aforementioned ocular fibrotic pathologies include loss of vision, blindness, mechanical disruption of the visual axis, opacification and decreased vision, or an otherwise impairment of visual function. In some embodiments, the present disclosure provides a method of reducing or ameliorating these symptoms. That is, in some embodiments, the present disclosure provides a method of increasing vision, maintaining of the visual axis in the eye, and preventing blindness.

Therapeutic Compounds

In some embodiments, a compound that can be used in any one of the methods described here is an agonist of a G protein coupled receptor. In such embodiments, the compound is a selective agonist of a Gas receptor (e.g., the compound is 100-fold, 50-fold, or 10-fold selective to Gas protein coupled receptor as compared to Gα_{12/13}, Gα_{q/11} or Gα_{i/o}, protein coupled receptor, or any combination of the aforementioned).

Since dopamine receptors are G protein coupled receptors, an agonist of a G protein coupled receptor agonizes a dopamine receptor (e.g., D1, D2, D3, D4, or D5 dopamine receptor). Hence, in some embodiments, a compound of the present disclosure is a dopamine receptor agonist.

Since dopamine D1 receptor and dopamine D5 receptor are both Ga s protein coupled receptors, an agonist of a Ga s protein coupled receptor agonizes either or both D1 and D5 receptors.

In some embodiments, a compound of the present disclosure is an agonist of a dopamine receptor D1 (DRD1). In some embodiments, the compound is a selective agonist of a dopamine receptor D1 (e.g., the compound is 100-fold, 50-fold, or 10-fold selective to D1 dopamine receptor as compared to D2, D3, D4, or D5 receptor, or any combination of the aforementioned). In some embodiments, the receptor agonist is a monoclonal or polyclonal antibody that is specific to dopamine receptor D1.

In some embodiments, a compound of the present disclosure is an agonist of a dopamine receptor D5 (DRD5). In some embodiments, the compound is a selective agonist of a dopamine receptor D5 (e.g., the compound is 100-fold, 50-fold, or 10-fold selective to D5 dopamine receptor as compared to D1, D2, D3, or D4 receptor, or any combination of the aforementioned). In some embodiments, the receptor agonist is a monoclonal or polyclonal antibody that is specific to dopamine receptor D5.

In some embodiments, a compound of the present disclosure is an agonist of a dopamine receptor D1 (DRD1) and a dopamine receptor D5 (DRD5). In some embodiments, the agonist is 100×, 50×, or 20× more selective for D1 receptor compared to D5 receptor. In some embodiments, the agonist is 100×, 50×, or 20× more selective for D5 receptor compared to D1 receptor. In one example, D5 is preferentially expressed in a cell over D1 (e.g., the cell comprises 100×, 50×, or 20× more D5 receptors than D1 receptors). In this example, the agonist is specific to D5 receptor over D1 receptor. In another example, D1 is preferentially expressed in a cell over D5 (e.g., the cell comprises 100×, 50×, or 20× more D1 receptors than D5 receptors). In this example, the agonist is specific to D1 receptor over D5 receptor.

In some embodiments, the compound of the present disclosure (e.g., a dopamine receptor agonist) is hydrophilic. In such embodiments, the structure of the compound contains hydrogen bond donor (HBD) atoms that are capable of forming hydrogen bonds with molecules of water and with the amino acids within the active site of the G protein coupled receptor. In some embodiments, the molecule of the receptor agonist contains at least 2, 3, 4, 5, or 6 HBD atoms (e.g., heteroatoms such as O, N or S). In some embodiments, the molecule of the receptor agonist contains at least one hydroxyl group (e.g., 1, 2, 3, 4, 5, or 6 hydroxyl groups). In some embodiments, the molecule of the receptor agonist contains amino groups (e.g., 1, 2, 3, 4, 5, or 6 amino groups).

In some embodiments, the compound does not penetrate the blood brain barrier or only an insignificant amount of the receptor agonist penetrates the blood brain barrier after the receptor agonist is administered to a subject (e.g., not more than about 0.1 wt. %, about 1 wt. %, about 5 wt. %, about 10 wt. %, or about 20 wt. % of the amount of the compound administered to the subject penetrates the blood brain barrier). In some embodiments, the compound (e.g., a dopamine receptor agonist) is ineffective or only weakly effective in treating central nervous system (CNS) disorders.

In some embodiments, the compound is a small molecule, e.g., about 2000 daltons or less (e.g., from about 300 to about 1200, from about 300 to about 1000, from about 300 to about 800, and/or from about 300 to about 600 daltons). In some embodiments, the compound is a biomolecule. Typically, biomolecules are organic molecules having a molecular weight of 200 daltons or more produced by living organisms or cells, including large polymeric molecules such as polypeptides, proteins, glycoproteins, polysaccharides, polynucleotides and nucleic acids. In some embodiments, the compound is an antibody, a hormone, a transmembrane protein, a growth factor, or an enzyme.

In some embodiments, the compound is selected from A-86929, dihydrexidine (DHX), dinapsoline, dinoxyline, doxanthrine, SKF-81297, SKF-82958, SKF-38393, fenoldopam, 6-Br-APB, stepholidine, A-68930, A-77636, CY-208,243, SKF-89145, SKF-89626, 7,8-dihydroxy-5-phenyl-octahydrobenzo[h]isoquinoline, cabergoline, and pergolide, or a pharmaceutically acceptable salt thereof.

In some embodiments, the compound is selected from ABT-413, A-86929, dihydrexidine (DHX), dinapsoline, dinoxyline, doxanthrine, SKF-81297, SKF-82958, SKF-38393, fenoldopam, 6-Br-APB, stepholidine, A-68930, A-77636, CY-208-243, SKF-89145, SKF-89626, 7,8-dihydroxy-5-phenyl-octahydrobenzo[h]isoquinoline, cabergoline, pergolide, R(−)-2,10,11-trihydroxyaporphine, (R)-(−)-apomorphine, R(−)-propylnorapomorphine, R(+)-6-bromo-APB, R(−)-2,10,11-trihydroxy-N-propyl-noraporphine, 6,7-ADTN, mesulergine, N-methyldopamine, 4-hydroxyphenethylamine, cabergoline, 3-hydroxyphenethylamine, pramipexole, PD-168077, fenoldopam, (±)-PD 128-907, (±)-2-(N-phenylethyl-N-propyl)amino-5-hydroxytetralin, bromocriptine, ropinirole, LY-163-502, dipropyldopamine, B-HT 920, piribedil, (+)-UH 232, pergolide, (−)-quinpirole, and R(−)-2,11-dihydroxy-10-methoxyapomorphine, or a pharmaceutically acceptable salt thereof.

In some embodiments, the compound is dihydrexidine (DHX), SKF-82958, A-68930, and CY-208-243, or a pharmaceutically acceptable salt thereof.

In some embodiments, the compound is dihydrexidine:

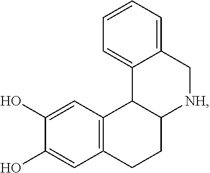 [SMILES: Oc1cc2c(cc1O)C1c3ccccc3CNC1CC2]

or a pharmaceutically acceptable salt thereof.

In some embodiments, the compound is A-68930:

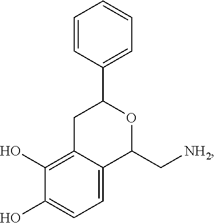 [SMILES: NCC1OC(c2ccccc2)Cc2c1ccc(O)c2O]

or a pharmaceutically acceptable salt thereof.

In some embodiments, the compound is SKF-82958:

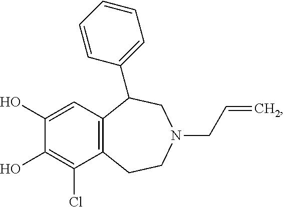 [SMILES: C=CCN1CCc2c(cc(O)c(O)c2Cl)C(c2ccccc2)C1]

or a pharmaceutically acceptable salt thereof.

In some embodiments, the compound is CY 208-243:

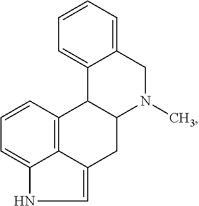 [SMILES: CN1Cc2ccccc2C2c3cccc4[nH]cc(c34)CC21]

or a pharmaceutically acceptable salt thereof

In some embodiments, the compound is SKF-38393:

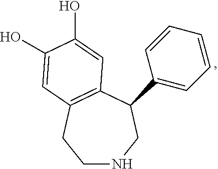 [SMILES: Oc1cc2c(cc1O)[C@@H](c1ccccc1)CNCC2 ; ","]

or a pharmaceutically acceptable salt thereof.

In some embodiments, the compound is A-77636:

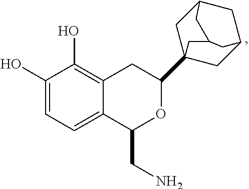 [SMILES: NC[C@@H]1O[C@H](C23CC4CC(CC(C4)C2)C3)Cc2c1ccc(O)c2O ; ","]

or a pharmaceutically acceptable salt thereof.

In some embodiments, the compound is A-86929:

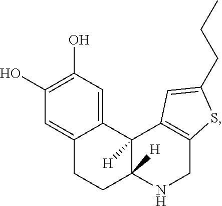

or a pharmaceutically acceptable salt thereof.

In some embodiments, the compound is ABT-431:

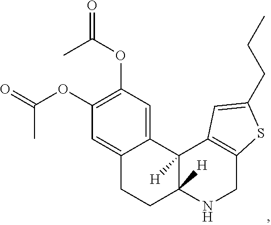

or a pharmaceutically acceptable salt thereof.

In some embodiments, the compound is any one of the compounds described in Martin et al., International Journal of Medicinal Chemistry, 2011, Article ID 424535, the disclosure of which is incorporated herein by reference in its entirety.

In some embodiments, the compound is a compound of Formula (I):

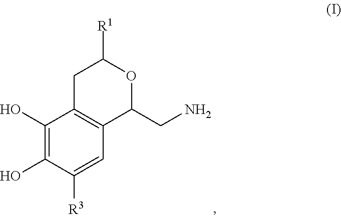

or a pharmaceutically acceptable salt thereof, wherein:
R¹ is selected from HO—C₁₋₆ alkyl, NH₂-C₁₋₆ alkyl, C₆₋₁₂ aryl ring, 5-6-membered heteroaryl ring comprising 1 to 5 heteroatoms selected from N, O, and S, and 3-10-membered heterocycloalkyl ring comprising 1 to 3 heteroatoms independently selected from N, O, and S;
wherein said C₆₋₁₂ aryl ring, heteroaryl ring, and heterocycloalkyl ring are each optionally substituted with 1, 2, or 3 substituents independently selected from R²;
each R² is independently selected from halo, OH, C₁₋₃ alkoxy, C₁₋₃ haloalkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino; and
R³ is selected from H and halo.

In some embodiments, the compound of Formula (I) has formula:

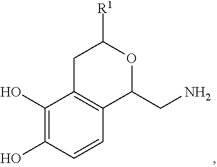

or a pharmaceutically acceptable salt thereof.
R¹ is selected from HO—C₁₋₆ alkyl, NH₂—C₁₋₆ alkyl, 5-6-membered heteroaryl ring comprising 1 to 5 heteroatoms selected from N, O, and S, and 3-10-membered heterocycloalkyl ring comprising 1 to 3 heteroatoms independently selected from N, O, and S;

In some embodiments, R¹ is C₆₋₁₂ aryl ring, which is optionally substituted with 1, 2, or 3 substituents independently selected from R².

In some embodiments, R¹ is a phenyl ring, which is optionally substituted with 1, 2, or 3 substituents independently selected from R².

In some embodiments, R¹ is a naphthyl ring, which is optionally substituted with 1, 2, or 3 substituents independently selected from R².

In some embodiments, the compound is:

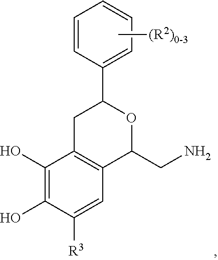

or a pharmaceutically acceptable salt thereof.

In some embodiments, R¹ is 5-6-membered heteroaryl ring comprising 1 to 5 heteroatoms selected from N, O, and S, which is optionally substituted with 1, 2, or 3 substituents independently selected from R².

In some embodiments, R¹ is 5-6-membered heteroaryl ring comprising 1 or 2 heteroatoms selected from N, O, and S, which is optionally substituted with 1, 2, or 3 substituents independently selected from R².

In some embodiments, R¹ is 5-membered heteroaryl ring comprising 1 or 2 heteroatoms selected from N, O, and S, which is optionally substituted with 1, 2, or 3 substituents independently selected from R².

In some embodiments, R¹ is selected from thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl, each of which is optionally substituted with 1, 2, or 3 substituents independently selected from R².

In some embodiments, R¹ is 6-membered heteroaryl ring comprising 1 or 2 N atoms, which is optionally substituted with 1, 2, or 3 substituents independently selected from R².

In some embodiments, R¹ is selected from pyridinyl, pyrimidinyl, pyrazinyl, diazinyl, triazinyl, tetrazinyl, and pentazinyl, each of which is optionally substituted with 1, 2, or 3 substituents independently selected from R.

In some embodiments, R¹ is selected from pyridyl, pyrazinyl, pyrimidinyl, and pyridazinyl, each of which is optionally substituted with 1, 2, or 3 substituents independently selected from R².

In some embodiments, R¹ is selected from pyridinyl, pyrimidinyl, and pyrazinyl, each of which is optionally substituted with 1, 2, or 3 substituents independently selected from R².

In some embodiments, R¹ is pyridinyl, optionally substituted with 1, 2, or 3 substituents independently selected from R².

In some embodiments, the compound of Formula (I) is:

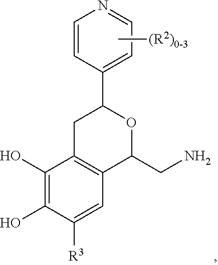

or a pharmaceutically acceptable salt thereof.

In some embodiments, the compound of Formula (I) is:

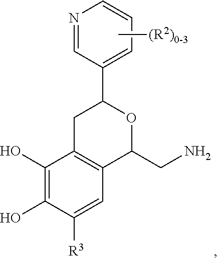

or a pharmaceutically acceptable salt thereof.

In some embodiments, the compound of Formula (I) is:

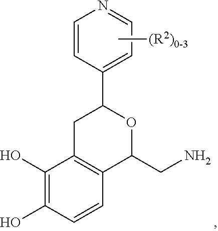

or a pharmaceutically acceptable salt thereof.

In some embodiments, the compound of Formula (I) is:

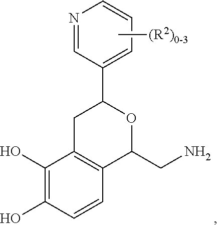

or a pharmaceutically acceptable salt thereof.

In some embodiments, R¹ is 3-10-membered heterocycloalkyl ring comprising 1 to 3 heteroatoms independently selected from N, O, and S, which is optionally substituted with 1, 2, or 3 substituents independently selected from R².

In some embodiments, R¹ is 3-7-membered heterocycloalkyl ring comprising 1 or 2 heteroatoms selected from N, O, and S, which is optionally substituted with 1, 2, or 3 substituents independently selected from R.

In some embodiments, R¹ is 3-membered heterocycloalkyl ring comprising 1 heteroatom selected from N, O, and S, which is optionally substituted with 1, 2, or 3 substituents independently selected from R². Examples of such rings include aziridinyl and oxiranyl.

In some embodiments, R¹ is 4-membered heterocycloalkyl ring comprising 1 heteroatom selected from N, O, and S, which is optionally substituted with 1, 2, or 3 substituents independently selected from R². Examples of such rings include oxetanyl and azetidinyl.

In some embodiments, R¹ is 5-membered heterocycloalkyl ring comprising 1 or 2 heteroatoms selected from N, O, and S, which is optionally substituted with 1, 2, or 3 substituents independently selected from R². Examples of such rings include tetrahydrofuranyl, pyrrolidinyl, isoxazolidinyl, imidazolidinyl, and thiazolidinyl.

In some embodiments, R¹ is 6-membered heterocycloalkyl ring comprising 1 or 2 heteroatoms selected from N, O, and S, which is optionally substituted with 1, 2, or 3 substituents independently selected from R². Examples of such rings include morpholinyl, thiomorpholinyl, tetrahydropyranyl, piperazinyl, and piperidinyl.

In some embodiments, R¹ is 7-membered heterocycloalkyl ring comprising 1 or 2 heteroatoms selected from N, O, and S, which is optionally substituted with 1, 2, or 3 substituents independently selected from R².

In some embodiments, R¹ is 8-membered heterocycloalkyl ring comprising 1 or 2 heteroatoms selected from N, O, and S, which is optionally substituted with 1, 2, or 3 substituents independently selected from R².

In some embodiments, R¹ is 9-membered heterocycloalkyl ring comprising 1 or 2 heteroatoms selected from N, O, and S, which is optionally substituted with 1, 2, or 3 substituents independently selected from R².

In some embodiments, R¹ is 10-membered heterocycloalkyl ring comprising 1 or 2 heteroatoms selected from N, O, and S, which is optionally substituted with 1, 2, or 3 substituents independently selected from R².

In some embodiments, R¹ is selected from tetrahydropyranyl, piperidinyl, tetrahydrofuranyl, pyrrolidinyl, pyranyl, morpholinyl, oxazinyl, dioxanyl, dioxinyl, diazinanyl, triazinanyl, trioxanyl, azepanyl, azepinyl, oxepanyl, oxepinyl, diazepanyl, diazepinyl, azocanyl, azocinyl, oxocanyl, oxocinyl, azonanyl, azoninyl, oxonanyl, and oxoninyl, each of which is optionally substituted with 1, 2, or 3 substituents independently selected from R².

In some embodiments, R¹ is selected from tetrahydropyranyl, piperidinyl, tetrahydrofuranyl, and pyrrolidinyl, each of which is optionally substituted with 1, 2, or 3 substituents independently selected from R².

In some embodiments, R¹ is selected from tetrahydropyranyl and piperidinyl, each of which is optionally substituted with 1, 2, or 3 substituents independently selected from R².

In some embodiments, R¹ is tetrahydropyranyl, optionally substituted with 1, 2, or 3 substituents independently selected from R.

In some embodiments, R¹ is piperidinyl, optionally substituted with 1, 2, or 3 substituents independently selected from R².

In some embodiments, the compound of Formula (I) is:

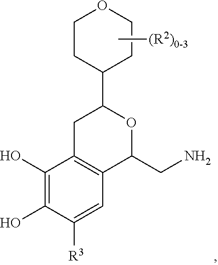

or a pharmaceutically acceptable salt thereof.

In some embodiments, the compound of Formula (I) is:

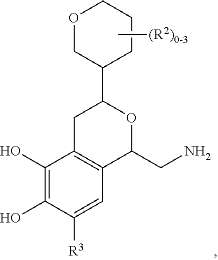

or a pharmaceutically acceptable salt thereof.

In some embodiments, the compound of Formula (I) is:

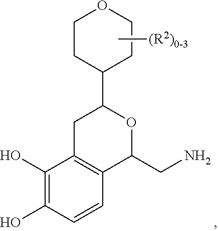

or a pharmaceutically acceptable salt thereof.

In some embodiments, the compound of Formula (I) is:

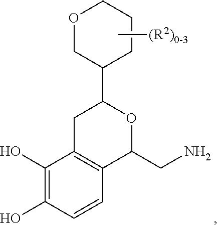

or a pharmaceutically acceptable salt thereof.

In some embodiments, the compound of Formula (I) is:

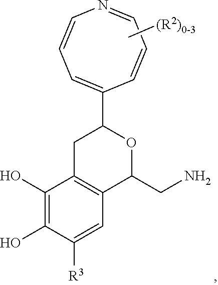

or a pharmaceutically acceptable salt thereof.

In some embodiments, the compound of Formula (I) is:

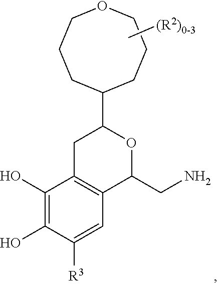

or a pharmaceutically acceptable salt thereof.

In some embodiments, R¹ is selected from HO—C₁₋₆ alkyl and NH₂—C₁₋₆ alkyl.

In some embodiments, R¹ is HO—C₁₋₆ alkyl.

In some embodiments, R¹ is NH₂—C₁₋₆ alkyl.

In some embodiments, R² is independently selected from halo, OH, C₁₋₃ alkoxy, C₁₋₃ alkyl, and C₁₋₃ haloalkyl.

In some embodiments, R² is selected from OH, NH₂, C₁₋₃ alkyl, HO—C₁₋₃ alkyl, and NH₂—C₁₋₃ alkyl. In some embodiments, R² is selected from OH and NH₂. In some embodiments, R² is OH. In some embodiments, R² is NH₂. In some embodiments, R² is C₁₋₃ alkyl. In some embodiments, R² is HO—C₁₋₃ alkyl. In some embodiments, R² is NH₂—C₁₋₃ alkyl.

In some embodiments, R³ is H.

In some embodiments, R³ is halo.

In some embodiments, R³ is selected from Cl, F, and Br. In some embodiments, R³ is Cl. In some embodiments, R³ is F. In some embodiments, R³ is Br.

In some embodiments, the compound of Formula (I) is selected from any one of the following compounds:

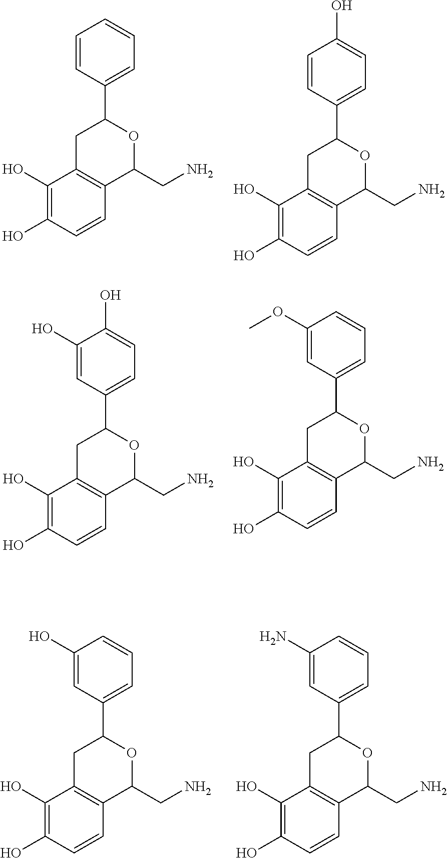 [SMILES: NCC1OC(c2ccccc2)Cc2c1ccc(O)c2O ; NCC1OC(c2ccc(O)cc2)Cc2c1ccc(O)c2O ; NCC1OC(c2ccc(O)c(O)c2)Cc2c1ccc(O)c2O ; COc1cccc(C2Cc3c(ccc(O)c3O)C(CN)O2)c1 ; NCC1OC(c2cccc(O)c2)Cc2c1ccc(O)c2O ; NCC1OC(c2cccc(N)c2)Cc2c1ccc(O)c2O]

or a pharmaceutically acceptable salt thereof.

In some embodiments, the compound of Formula (I) is selected from any one of the following compounds:

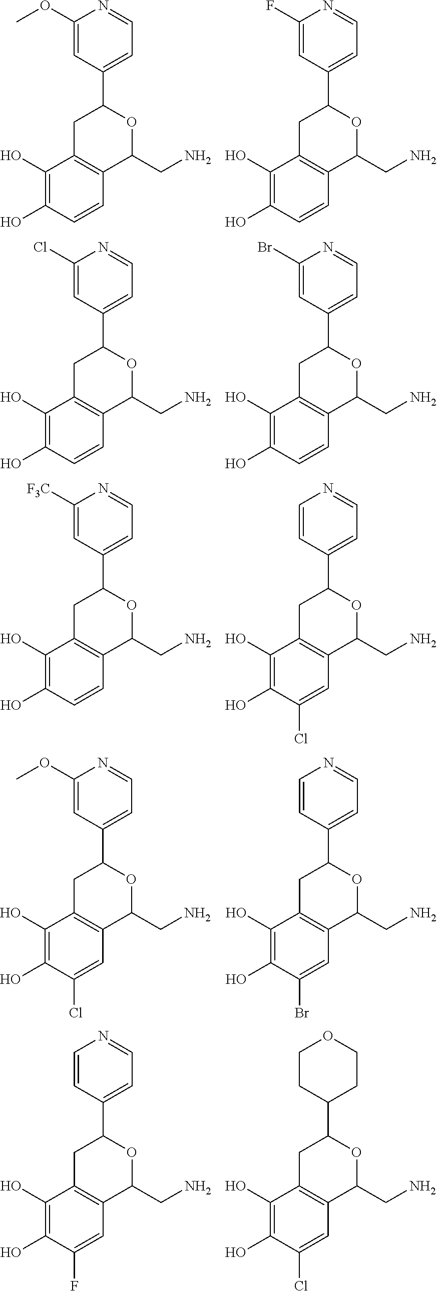

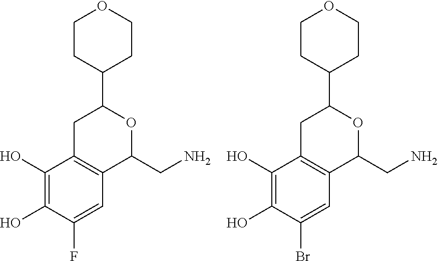 [SMILES: NCC1OC(C2CCOCC2)Cc2c1cc(F)c(O)c2O ; NCC1OC(C2CCOCC2)Cc2c1cc(Br)c(O)c2O]

or a pharmaceutically acceptable salt thereof.

In some embodiments, the compound of Formula (I) is selected from any one of the following compounds:

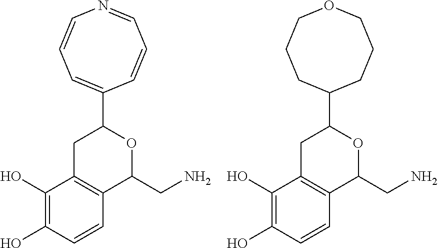 [SMILES: NCC1OC(C2=C/C=C\N=C/C=C\2)Cc2c1ccc(O)c2O ; NCC1OC(C2CCCOCCC2)Cc2c1ccc(O)c2O]

or a pharmaceutically acceptable salt thereof.

In some embodiments, the compound of Formula (I) is selected from any one of the following compounds:

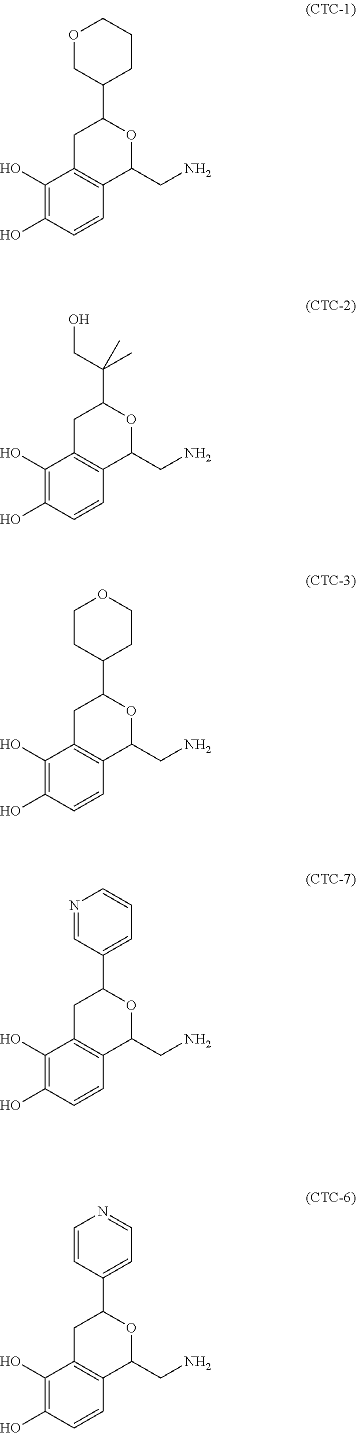 [SMILES: NCC1OC(C2CCCOC2)Cc2c1ccc(O)c2O ; CC(C)(CO)C1Cc2c(ccc(O)c2O)C(CN)O1 ; NCC1OC(C2CCOCC2)Cc2c1ccc(O)c2O ; NCC1OC(c2cccnc2)Cc2c1ccc(O)c2O ; NCC1OC(c2ccncc2)Cc2c1ccc(O)c2O]

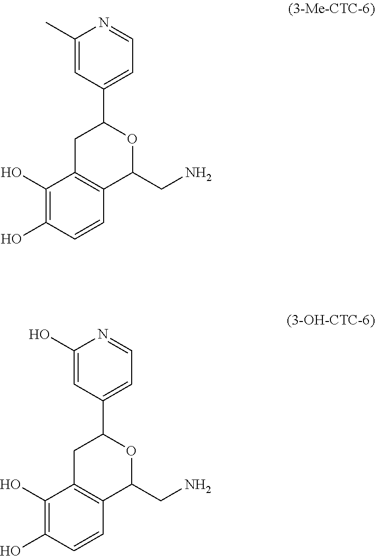 [SMILES: Cc1cc(C2Cc3c(ccc(O)c3O)C(CN)O2)ccn1 ; NCC1OC(c2ccnc(O)c2)Cc2c1ccc(O)c2O]

or a pharmaceutically acceptable salt thereof.

In some embodiments, the compound useful in the methods of this disclosure is a compound of Formula (II):

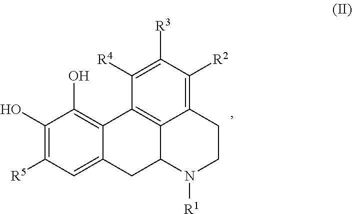

or a pharmaceutically acceptable salt thereof, wherein:
R¹ is selected from H and C₁₋₃ alkyl, wherein said C₁₋₃ alkyl is optionally substituted with OH, SH, NH₂, C₁₋₃ alkylamino, or di(C₁₋₃ alkyl)amino;
R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, SH, NH₂, C₁₋₃ alkylamino, and di(C₁₋₃ alkyl)amino; and
R⁵ is selected from H and halo.

In some embodiments, the compound of Formula (II) has formula:

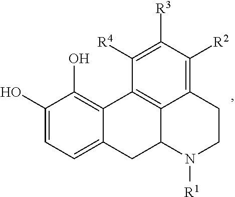

or a pharmaceutically acceptable salt thereof.

In some embodiments, R¹ is H.

In some embodiments, R¹ is C₁₋₃ alkyl (e.g., methyl, ethyl, propyl, isopropyl).

In some embodiments, R¹ is selected from HO—C₁₋₃ alkyl and NH₂—C₁₋₃ alkyl.

In some embodiments, R¹ is HO—C₁₋₃ alkyl. In some embodiments, R¹ is NH₂-C₁₋₃ alkyl.

In some embodiments, at least one of R², R³, and R⁴ is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, at least one of R², R³, and R⁴ is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.

In some embodiments, at least one of R², R³, and R⁴ is selected from NH₂, C₁₋₃ alkyl, HO—C₁₋₃ alkyl, and NH₂—C₁₋₃ alkyl.

In some embodiments, at least one of R², R³, and R⁴ is C₁₋₃ alkyl.

In some embodiments, R² is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R² is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R² is selected from OH, NH₂, C₁₋₃ alkyl, HO—C₁₋₃ alkyl, and NH₂—C₁₋₃ alkyl. In some embodiments, R² is selected from NH₂, C₁₋₃ alkyl, HO—C₁₋₃ alkyl, and NH₂—C₁₋₃ alkyl. In some embodiments, R² is selected from C₁₋₃ alkyl, HO—C₁₋₃ alkyl, and NH₂—C₁₋₃ alkyl. In some embodiments, R² is selected from C₁₋₃ alkyl and HO—C₁₋₃ alkyl. In some embodiments, R² is C₁₋₃ alkyl (e.g., methyl, ethyl, propyl, isopropyl). In some embodiments, R² is HO—C₁₋₃ alkyl. In some embodiments, R² is NH₂—C₁₋₃ alkyl. In some embodiments, R² is OH. In some embodiments, R² is NH₂. In some embodiments, R² is H.

In some embodiments, R³ is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R³ is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R³ is selected from OH, NH₂, C₁₋₃ alkyl, HO—C₁₋₃ alkyl, and NH₂—C₁₋₃ alkyl. In some embodiments, R³ is selected from NH₂, C₁₋₃ alkyl, HO—C₁₋₃ alkyl, and NH₂—C₁₋₃ alkyl. In some embodiments, R³ is selected from C₁₋₃ alkyl, HO—C₁₋₃ alkyl, and NH₂—C₁₋₃ alkyl. In some embodiments, R³ is selected from C₁₋₃ alkyl and HO—C₁₋₃ alkyl. In some embodiments, R³ is C₁₋₃ alkyl (e.g., methyl, ethyl, propyl, isopropyl). In some embodiments, R³ is HO—C₁₋₃ alkyl. In some embodiments, R³ is NH₂—C₁₋₃ alkyl. In some embodiments, R³ is OH. In some embodiments, R³ is NH₂. In some embodiments, R³ is H.

In some embodiments, R⁴ is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R⁴ is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R⁴ is selected from OH, NH₂, C₁₋₃ alkyl, HO—C₁₋₃ alkyl, and NH₂-C₁₋₃ alkyl. In some embodiments, R⁴ is selected from NH₂, C₁₋₃ alkyl, HO—C₁₋₃ alkyl, and NH₂—C₁₋₃ alkyl. In some embodiments, R⁴ is selected from C₁₋₃ alkyl, HO—C₁₋₃ alkyl, and NH₂—C₁₋₃ alkyl. In some embodiments, R⁴ is selected from C₁₋₃ alkyl and HO—C₁₋₃ alkyl. In some embodiments, R⁴ is C₁₋₃ alkyl (e.g., methyl, ethyl, propyl, isopropyl). In some embodiments, R⁴ is HO—C₁₋₃ alkyl. In some embodiments, R⁴ is NH₂—C₁₋₃ alkyl. In some embodiments, R⁴ is OH. In some embodiments, R⁴ is NH₂. In some embodiments, R⁴ is H.

In some embodiments:
R³ is OH; and
R² is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.

In some embodiments:
R³ is OH; and
R² is selected from OH, NH₂, C₁₋₃ alkyl, HO—C₁₋₃ alkyl, and NH₂—C₁₋₃ alkyl.

In some embodiments:
R³ is OH; and
R⁴ is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.

In some embodiments:
R³ is OH; and
R⁴ is selected from NH₂, C₁₋₃ alkyl, HO—C₁₋₃ alkyl, and NH₂—C₁₋₃ alkyl.

In some embodiments:
R⁴ is OH; and
R³ is selected from H, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.

In some embodiments:
R⁴ is OH; and
R³ is selected from H, NH₂, C₁₋₃ alkyl, HO—C₁₋₃ alkyl, and NH₂—C₁₋₃ alkyl.

In some embodiments:
R² is OH; and
at least one of R³ and R⁴ is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.

In some embodiments:
R² is OH; and
at least one of R³ and R⁴ is selected from OH, NH₂, C₁₋₃ alkyl, HO—C₁₋₃ alkyl, and NH₂—C₁₋₃ alkyl.

In some embodiments, R³ is OH and R² is C₁₋₃ alkyl. In some embodiments, R³ is C₁₋₃ alkyl and R² is OH. In some embodiments, R³ is OH and R⁴ is C₁₋₃ alkyl. In some embodiments, R³ is C₁₋₃ alkyl and R⁴ is OH.

In some embodiments:
R⁵ is halo; and
R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, SH, NH₂, C₁₋₃ alkylamino, and di(C₁₋₃ alkyl)amino.

In some embodiments, R², R³, and R⁴ are each independently selected from H, OH, and C₁₋₃ alkyl. In some embodiments, R², R³, and R⁴ are each H.

In some embodiments, R⁵ is H. In some embodiments, R⁵ is halo. In some embodiments, R⁵ is selected from Cl, Br, and F. In some embodiments, R⁵ is Cl. In some embodiments, R⁵ is Br. In some embodiments, R⁵ is F.

In some embodiments, the compound of Formula (II) is:

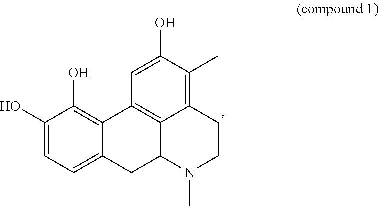 [SMILES: Cc1c(O)cc2c3c1CCN(C)C3Cc1ccc(O)c(O)c1-2]

or a pharmaceutically acceptable salt thereof.

In some embodiments, the compound of Formula (II) is:

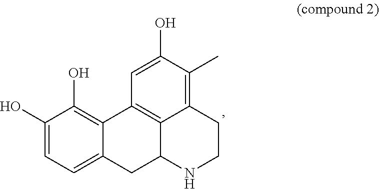 [SMILES: Cc1c(O)cc2c3c1CCNC3Cc1ccc(O)c(O)c1-2]

or a pharmaceutically acceptable salt thereof.

In some embodiments, the compound of Formula (II) is selected from any one of the following compounds:

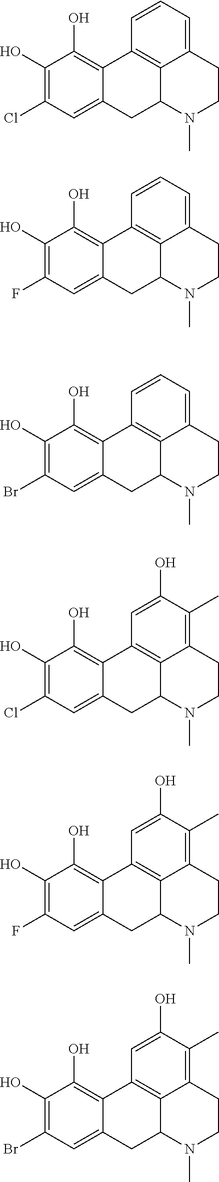

or a pharmaceutically acceptable salt thereof.

In some embodiments, the compound useful in the methods of this disclosure is a compound of Formula (III):

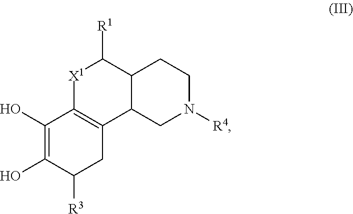

or a pharmaceutically acceptable salt thereof, wherein:
X¹ is selected from CH₂ and O;
R¹ is selected from HO—C₁₋₆ alkyl, NH₂—C₁₋₆ alkyl, C₆₋₁₂ aryl ring, 5-6-membered heteroaryl ring comprising 1 to 5 heteroatoms selected from N, O, and S, and 3-10-membered heterocycloalkyl ring comprising 1 to 3 heteroatoms independently selected from N, O, and S;
wherein said aryl ring, heteroaryl ring, and heterocycloalkyl ring are each optionally substituted with 1, 2, or 3 substituents independently selected from R²;
each R² is independently selected from halo, OH, C₁₋₃ alkoxy, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, C₁₋₃ alkoxy, SH, NH₂, C₁₋₃ alkylamino, and di(C₁₋₃ alkyl)amino;
R³ is selected from H and halo; and
R⁴ is selected from H and C₁₋₃ alkyl, wherein said C₁₋₃ alkyl is optionally substituted with OH, SH, NH₂, C₁₋₃ alkylamino, or di(C₁₋₃ alkyl)amino.

In some embodiments, X¹ is CH₂. In some embodiments, X¹ is O.

In some embodiments, R³ is H. In some embodiments, R³ is halo. In some embodiments, R³ is selected from Cl, Br, and F. In some embodiments, R³ is Cl. In some embodiments, R³ is Br. In some embodiments, R³ is F.

In some embodiments, R⁴ is selected from H and C₁₋₃ alkyl.

In some embodiments, R⁴ is H. In some embodiments, R⁴ is C₁₋₃ alkyl.

R¹ is selected from HO—C₁₋₆ alkyl, NH₂—C₁₋₆ alkyl, 5-6-membered heteroaryl ring comprising 1 to 5 heteroatoms selected from N, O, and S, and 3-10-membered heterocycloalkyl ring comprising 1 to 3 heteroatoms independently selected from N, O, and S; In some embodiments, R¹ is C₆₋₁₂ aryl ring, which is optionally substituted with 1, 2, or 3 substituents independently selected from R².

In some embodiments, R¹ is a phenyl ring, which is optionally substituted with 1, 2, or 3 substituents independently selected from R².

In some embodiments, R¹ is a naphthyl ring, which is optionally substituted with 1, 2, or 3 substituents independently selected from R².

In some embodiments, the compound of Formula (III) has formula:

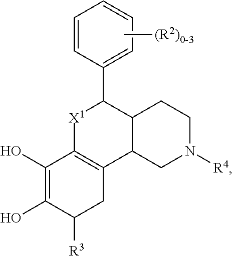

or a pharmaceutically acceptable salt thereof.

In some embodiments, R¹ is 5-6-membered heteroaryl ring comprising 1 to 5 heteroatoms selected from N, O, and S, which is optionally substituted with 1, 2, or 3 substituents independently selected from R.

In some embodiments, R¹ is 5-6-membered heteroaryl ring comprising 1 or 2 heteroatoms selected from N, O, and S, which is optionally substituted with 1, 2, or 3 substituents independently selected from R.

In some embodiments, R¹ is 5-membered heteroaryl ring comprising 1 or 2 heteroatoms selected from N, O, and S, which is optionally substituted with 1, 2, or 3 substituents independently selected from R.

In some embodiments, R¹ is selected from thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl, each of which is optionally substituted with 1, 2, or 3 substituents independently selected from R².

In some embodiments, R¹ is 6-membered heteroaryl ring comprising 1 or 2 N atoms, which is optionally substituted with 1, 2, or 3 substituents independently selected from R².

In some embodiments, R¹ is selected from pyridinyl, pyrimidinyl, pyrazinyl, diazinyl, triazinyl, tetrazinyl, and pentazinyl, each of which is optionally substituted with 1, 2, or 3 substituents independently selected from R².

In some embodiments, R¹ is selected from pyridyl, pyrazinyl, pyrimidinyl, and pyridazinyl, each of which is optionally substituted with 1, 2, or 3 substituents independently selected from R.

In some embodiments, R¹ is selected from pyridinyl, pyrimidinyl, and pyrazinyl, each of which is optionally substituted with 1, 2, or 3 substituents independently selected from R.

In some embodiments, R¹ is pyridinyl, optionally substituted with 1, 2, or 3 substituents independently selected from R².

In some embodiments, the compound of Formula (III) has formula:

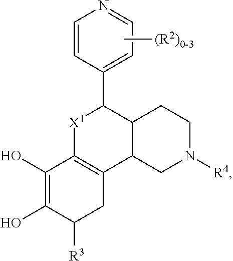

or a pharmaceutically acceptable salt thereof.

In some embodiments, the compound of Formula (III) has formula:

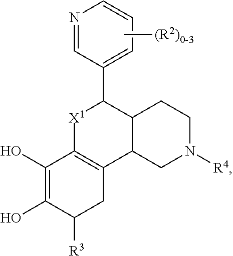

or a pharmaceutically acceptable salt thereof.

In some embodiments, R¹ is 3-10-membered heterocycloalkyl ring comprising 1 to 3 heteroatoms independently selected from N, O, and S, which is optionally substituted with 1, 2, or 3 substituents independently selected from R.

In some embodiments, R¹ is 3-7-membered heterocycloalkyl ring comprising 1 or 2 heteroatoms selected from N, O, and S, which is optionally substituted with 1, 2, or 3 substituents independently selected from R.

In some embodiments, R¹ is 3-membered heterocycloalkyl ring comprising 1 heteroatom selected from N, O, and S, which is optionally substituted with 1, 2, or 3 substituents independently selected from R². Examples of such rings include aziridinyl and oxiranyl.

In some embodiments, R¹ is 4-membered heterocycloalkyl ring comprising 1 heteroatom selected from N, O, and S, which is optionally substituted with 1, 2, or 3 substituents independently selected from R². Examples of such rings include oxetanyl and azetidinyl.

In some embodiments, R¹ is 5-membered heterocycloalkyl ring comprising 1 or 2 heteroatoms selected from N, O, and S, which is optionally substituted with 1, 2, or 3 substituents independently selected from R². Examples of such rings include tetrahydrofuranyl, pyrrolidinyl, isoxazolidinyl, imidazolidinyl, and thiazolidinyl.

In some embodiments, R¹ is 6-membered heterocycloalkyl ring comprising 1 or 2 heteroatoms selected from N, O, and S, which is optionally substituted with 1, 2, or 3 substituents independently selected from R². Examples of such rings include morpholinyl, thiomorpholinyl, tetrahydropyranyl, piperazinyl, and piperidinyl.

In some embodiments, R¹ is 7-membered heterocycloalkyl ring comprising 1 or 2 heteroatoms selected from N, O, and S, which is optionally substituted with 1, 2, or 3 substituents independently selected from R².

In some embodiments, R¹ is 8-membered heterocycloalkyl ring comprising 1 or 2 heteroatoms selected from N, O, and S, which is optionally substituted with 1, 2, or 3 substituents independently selected from R².

In some embodiments, R¹ is 9-membered heterocycloalkyl ring comprising 1 or 2 heteroatoms selected from N, O, and S, which is optionally substituted with 1, 2, or 3 substituents independently selected from R².

In some embodiments, R¹ is 10-membered heterocycloalkyl ring comprising 1 or 2 heteroatoms selected from N, O, and S, which is optionally substituted with 1, 2, or 3 substituents independently selected from R².

In some embodiments, R¹ is selected from tetrahydropyranyl, piperidinyl, tetrahydrofuranyl, pyrrolidinyl, pyranyl, morpholinyl, oxazinyl, dioxanyl, dioxinyl, diazinanyl, triazinanyl, trioxanyl, azepanyl, azepinyl, oxepanyl, oxepinyl, diazepanyl, diazepinyl, azocanyl, azocinyl, oxocanyl, oxocinyl, azonanyl, azoninyl, oxonanyl, and oxoninyl, each of which is optionally substituted with 1, 2, or 3 substituents independently selected from R².

In some embodiments, R¹ is selected from tetrahydropyranyl, piperidinyl, tetrahydrofuranyl, and pyrrolidinyl, each of which is optionally substituted with 1, 2, or 3 substituents independently selected from R².

In some embodiments, R¹ is selected from tetrahydropyranyl and piperidinyl, each of which is optionally substituted with 1, 2, or 3 substituents independently selected from R².

In some embodiments, R¹ is tetrahydropyranyl, optionally substituted with 1, 2, or 3 substituents independently selected from R². In some embodiments, R¹ is any one of R¹ described herein for Formula (I).

In some embodiments, the compound of Formula (III) has formula:

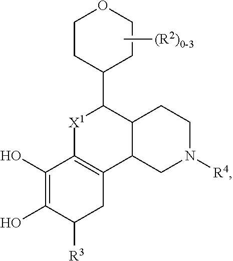

or a pharmaceutically acceptable salt thereof.

In some embodiments, the compound of Formula (III) has formula:

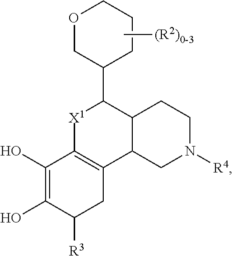

or a pharmaceutically acceptable salt thereof.

In some embodiments, R¹ is selected from HO—C₁₋₆ alkyl and NH₂—C₁₋₆ alkyl.

In some embodiments, R¹ is HO—C₁₋₆ alkyl.

In some embodiments, R¹ is NH₂—C₁₋₆ alkyl.

In some embodiments, each R² is independently selected from halo, OH, C₁₋₃ alkoxy, C₁₋₃ alkyl, and C₁₋₃ haloalkyl. In some embodiments, R² is any of the R² groups described herein for the compound of Formula (I). In some embodiments, R² is selected from OH, NH₂, C₁₋₃ alkyl, HO—C₁₋₃ alkyl, and NH₂—C₁₋₃ alkyl. In some embodiments, R² is selected from OH and NH₂. In some embodiments, R² is OH. In some embodiments, R² is NH₂. In some embodiments, R² is C₁₋₃ alkyl. In some embodiments, R² is HO—C₁₋₃ alkyl. In some embodiments, R² is NH₂-C₁₋₃ alkyl.

In some embodiments, the compound of Formula (III) is selected from any one of the following compounds:

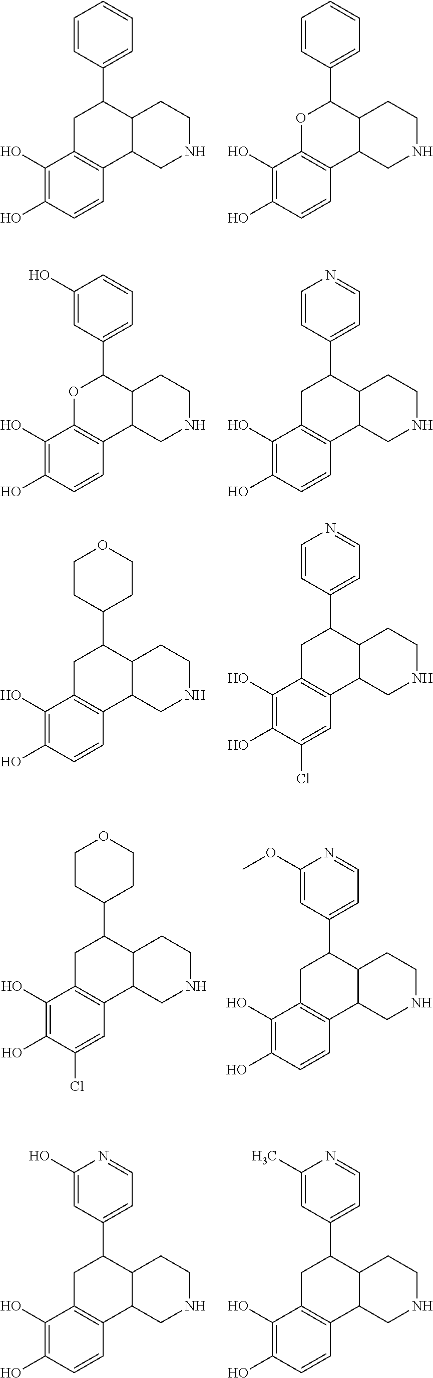

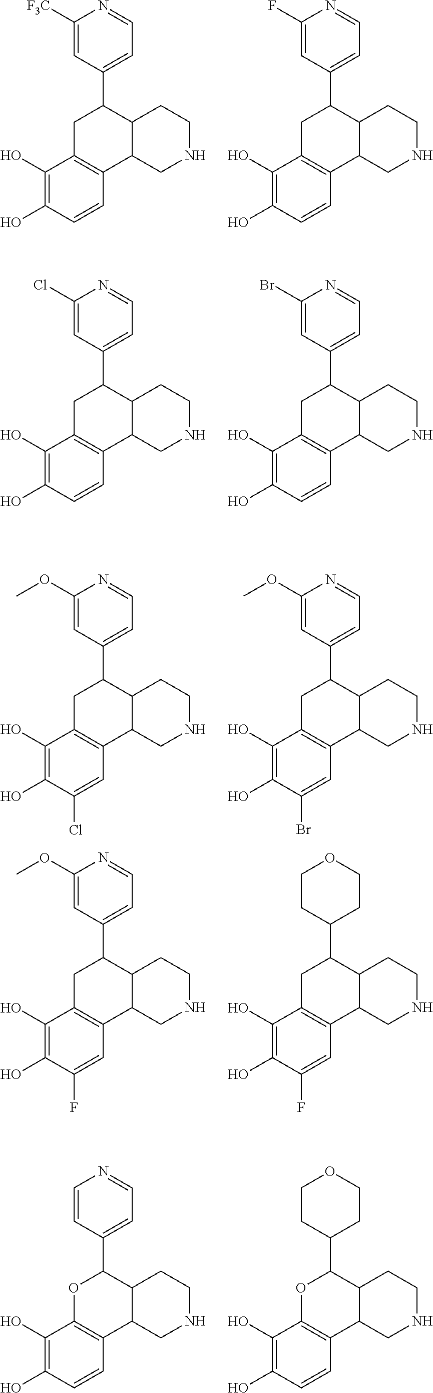

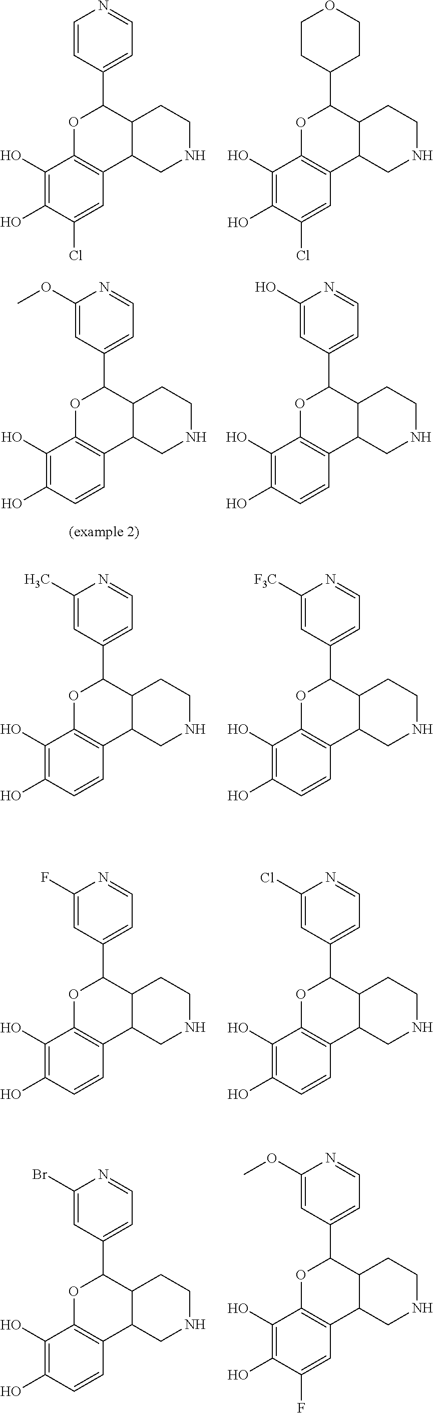

or a pharmaceutically acceptable salt thereof.

In some embodiments, the compound useful in the methods of this disclosure is a compound of Formula (IV):

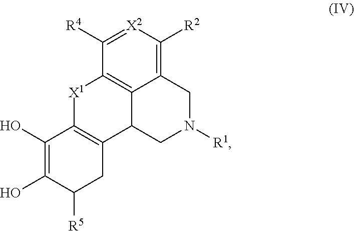

or a pharmaceutically acceptable salt thereof, wherein:
X¹ is selected from CH₂ and O;
X² is selected from CR³ and N;
R¹ is selected from H and C₁₋₃ alkyl, wherein said C₁₋₃ alkyl is optionally substituted with OH, SH, NH₂, C₁₋₃ alkylamino, or di(C₁₋₃ alkyl)amino;
R⁵ is selected from H and halo; and
R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, SH, NH₂, C₁₋₃ alkylamino, and di(C₁₋₃ alkyl)amino.

In some embodiments of the compound of Formula (IV), if R⁵ is H and X² is CR³, then at least one of R², R³, and R⁴ is not H.

In some embodiments, X¹ is CH₂. In some embodiments, X¹ is O.

In some embodiments, R¹ is selected from H and C₁₋₃ alkyl.

In some embodiments, R¹ is H. In some embodiments, R¹ is C₁₋₃ alkyl (e.g., methyl, ethyl, propyl, isopropyl). In some embodiments, R¹ is selected from HO—C₁₋₃ alkyl and NH₂—C₁₋₃ alkyl. In some embodiments, R¹ is HO—C₁₋₃ alkyl. In some embodiments, R¹ is NH₂-C₁₋₃ alkyl.

In some embodiments, R⁵ is H. In some embodiments, R⁵ is halo. In some embodiments, R⁵ is selected from Cl, Br, and F. In some embodiments, R⁵ is Cl. In some embodiments, R⁵ is Br. In some embodiments, R⁵ is F.

In some embodiments, X² is N. In some embodiments, X² is CR³.

In some embodiments, at least one of R², R³, and R⁴ is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, at least one of R², R³, and R⁴ is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.

In some embodiments, at least one of R², R³, and R⁴ is selected from NH₂, C₁₋₃ alkyl, HO—C₁₋₃ alkyl, and NH₂—C₁₋₃ alkyl.

In some embodiments, at least one of R², R³, and R⁴ is C₁₋₃ alkyl.

In some embodiments, R² is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R² is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R² is selected from OH, NH₂, C₁₋₃ alkyl, HO—C₁₋₃ alkyl, and NH₂—C₁₋₃ alkyl. In some embodiments, R² is selected from NH₂, C₁₋₃ alkyl, HO—C₁₋₃ alkyl, and NH₂—C₁₋₃ alkyl. In some embodiments, R² is selected from C₁₋₃ alkyl, HO—C₁₋₃ alkyl, and NH₂—C₁₋₃ alkyl. In some embodiments, R² is selected from C₁₋₃ alkyl and HO—C₁₋₃ alkyl. In some embodiments, R² is C₁₋₃ alkyl (e.g., methyl, ethyl, propyl, isopropyl). In some embodiments, R² is HO—C₁₋₃ alkyl. In some embodiments, R² is NH₂—C₁₋₃ alkyl. In some embodiments, R² is OH. In some embodiments, R² is NH₂. In some embodiments, R² is H.

In some embodiments, R³ is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R³ is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R³ is selected from OH, NH₂, C₁₋₃ alkyl, HO—C₁₋₃ alkyl, and NH₂—C₁₋₃ alkyl. In some embodiments, R³ is selected from NH₂, C₁₋₃ alkyl, HO—C₁₋₃ alkyl, and NH₂—C₁₋₃ alkyl. In some embodiments, R³ is selected from C₁₋₃ alkyl, HO—C₁₋₃ alkyl, and NH₂—C₁₋₃ alkyl. In some embodiments, R³ is selected from C₁₋₃ alkyl and HO—C₁₋₃ alkyl. In some embodiments, R³ is C₁₋₃ alkyl (e.g., methyl, ethyl, propyl, isopropyl). In some embodiments, R³ is HO—C₁₋₃ alkyl. In some embodiments, R³ is NH₂—C₁₋₃ alkyl. In some embodiments, R³ is OH. In some embodiments, R³ is NH₂. In some embodiments, R³ is H.

In some embodiments, R⁴ is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R⁴ is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R⁴ is selected from OH, NH₂, C₁₋₃ alkyl, HO—C₁₋₃ alkyl, and NH₂-C₁₋₃ alkyl. In some embodiments, R⁴ is selected from NH₂, C₁₋₃ alkyl, HO—C₁₋₃ alkyl, and NH₂—C₁₋₃ alkyl. In some embodiments, R⁴ is selected from C₁₋₃ alkyl, HO—C₁₋₃ alkyl, and NH₂—C₁₋₃ alkyl. In some embodiments, R⁴ is selected from C₁₋₃ alkyl and HO—C₁₋₃ alkyl. In some embodiments, R⁴ is C₁₋₃ alkyl (e.g., methyl, ethyl, propyl, isopropyl). In some embodiments, R⁴ is HO—C₁₋₃ alkyl. In some embodiments, R⁴ is NH₂—C₁₋₃ alkyl. In some embodiments, R⁴ is OH. In some embodiments, R⁴ is NH₂. In some embodiments, R⁴ is H.

In some embodiments, the compound of Formula (IV) has formula:

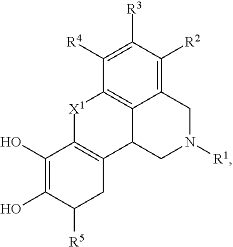

or a pharmaceutically acceptable salt thereof.

In some embodiments:
R⁵ is halo; and
R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.

In some embodiments, R², R³, and R⁴ are each independently selected from H, OH, and C₁₋₃ alkyl. In some embodiments, R², R³, and R⁴ are each H.

In some embodiments:
R³ is H;
R⁵ is H; and
R² and R⁴ are each independently selected from OH and C₁₋₃ alkyl.

In some embodiments, the compound of Formula (IV) has formula:

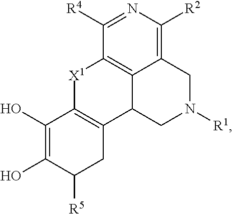

or a pharmaceutically acceptable salt thereof.

In some embodiments, R² and R⁴ are each independently selected from H, halo, OH, C₁₋₃ alkoxy, C₁₋₃ alkyl, and C₁₋₃ haloalkyl.

In some embodiments, the compound of Formula (IV) is selected from any one of the following compounds:

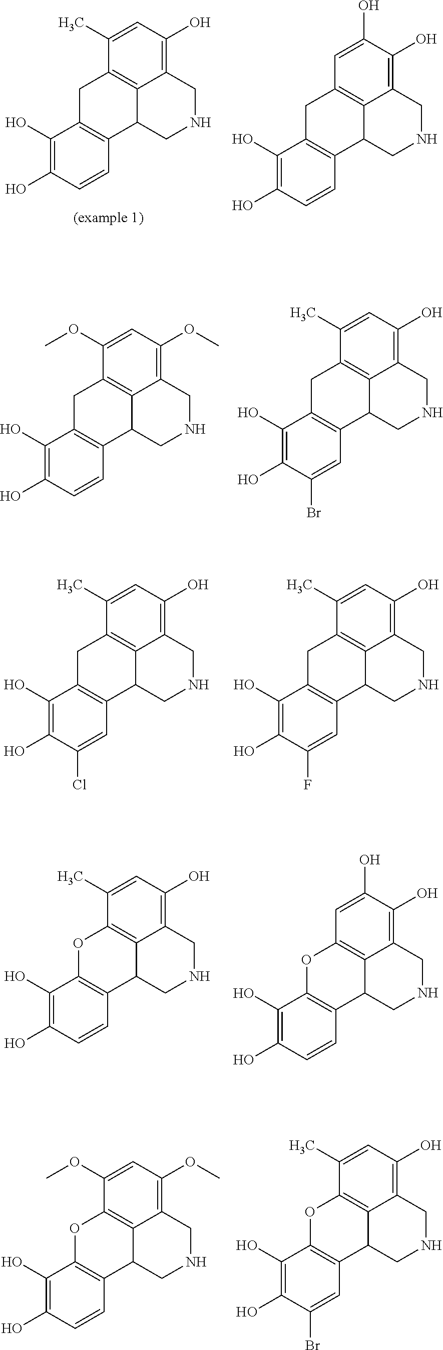

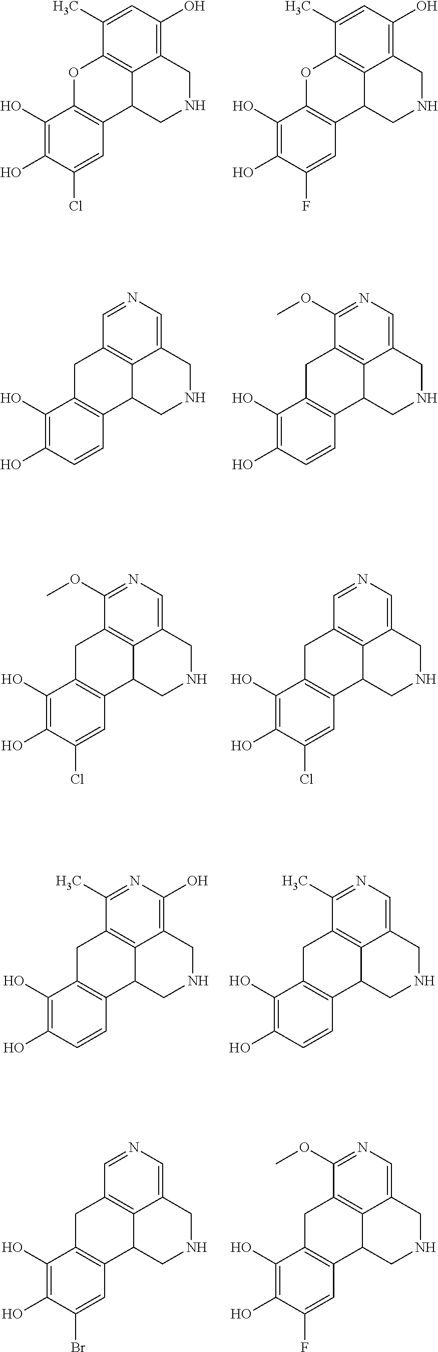

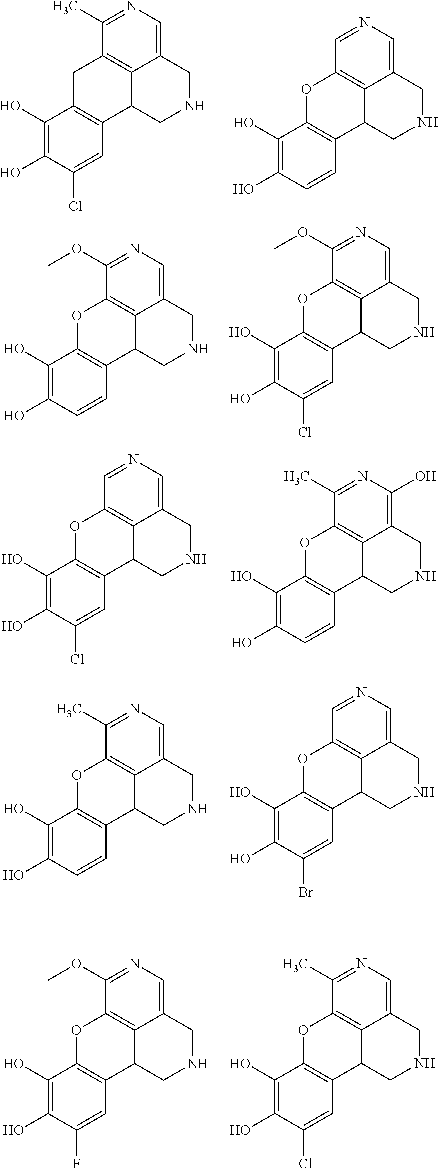

or a pharmaceutically acceptable salt thereof.

Pharmaceutical Compositions

The present application also provides pharmaceutical compositions comprising an effective amount of a compound of the present disclosure, or a pharmaceutically acceptable salt thereof, and a pharmaceutically acceptable carrier. The pharmaceutical composition may also comprise at least one of any one of the additional therapeutic agents described. In certain embodiments, the application also provides pharmaceutical compositions and dosage forms comprising any one the additional therapeutic agents described herein (e.g., in a kit). The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral, vaginal, and ocular.

Compositions and formulations described herein may conveniently be presented in a unit dosage form, e.g., tablets, capsules (e.g., hard or soft gelatin capsules), sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

In some embodiments, the present disclosure provides a pharmaceutical formulation of a compound described herein that is suitable for ocular (topical) administration (e.g., administration by an ocular route). Suitable examples of such formulations include eye-drops, eye ointment, and eye emulsion. The formulation contains additional ingredients that allow the compound to permeate into main ocular circulatory system and cross the ocular barrier. In some embodiments, the compound can be coated on a contact lens. In some embodiments, the compound can be administered by a local injection into or near the cornea, choroid, retina, vitreous, uvea, orbit, eyelid, conjunctiva, or iris. Suitable examples of intraocular routes include: intravitreal, intraocular, intracameral, subconjunctival, subtenon, intracorneal, intrastromal, trans-scleral, and suprachoroidal route. Any of the formulations described herein can be administered by any of these routes. The compound can be coated on any implant, stent or drainage device placed in or around the eye or orbit. The compound can be coated on a contact lens or scleral lens or a punctal plug. The compound can also be made into a sustained release delivery device on any of the aforementioned routes or devices. The compound can be topical eye drops or gels or coated on cotton tips or Weck-cells or similar applicators on the surface of the eye. The compound can be delivered by nanop article delivery devices or ultrasound or electrical stimulation. The compound could be light activated or activated or delivered by any known delivery route currently available.

Dosages and Regimens

In the pharmaceutical compositions of the present application, a therapeutic compound is present in an effective amount (e.g., a therapeutically effective amount).

Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a therapeutic compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0. 1 mg/kg to about 200 mg/kg; from about 0. 1 mg/kg to about 150 mg/kg; from about 0. 1 mg/kg to about 100 mg/kg; from about 0.1 mg/kg to about 50 mg/kg; from about 0. 1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of a therapeutic compound is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month). The compounds and compositions described herein can be administered to the subject in any order. A first therapeutic agent, such as a compound of the present disclosure, can be administered prior to or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before or after), or concomitantly with the administration of a second therapeutic agent, such as an anti-fibrotic agent described herein, to a subject in need of treatment. Thus, the compound of the present disclosure, or a composition containing the compound, can be administered separately, sequentially or simultaneously with the second therapeutic agent, such as an anti-fibrotic agent described herein. When the compound of the present disclosure, or a pharmaceutically acceptable salt thereof, and a second or third therapeutic agent are administered to the subject simultaneously, the therapeutic agents may be administered in a single dosage form (e.g., tablet, capsule, or a solution for injection or infusion).

In some embodiments, the second (additional) therapeutic agent is a drug that is useful in treating or preventing an ocular fibrotic pathology. In some embodiments, the additional therapeutic agent is dopamine, or a pharmaceutically acceptable salt thereof. In some embodiments, the additional therapeutic agent is a dopamine receptor agonist. In some embodiments, the second (additional) therapeutic agent is an anti-inflammatory drug. Suitable examples of such drugs include NSAIDs such as celecoxib, rofecoxib, ibuprofen, naproxen, aspirin, diclofenac, sulindac, oxaprozin, piroxicam, indomethacin, meloxicam, fenoprofen, diflunisal, methotrexate, BAY 11-7082, or a pharmaceutically acceptable salt thereof. Suitable examples of steroid anti-inflammatory agents include cortisol, corticosterone, hydrocortisone, aldosterone, deoxycorticosterone, triamcinolone, bardoxolone, bardoxolone methyl, triamcinolone, cortisone, prednisone, and methylprednisolone, or a pharmaceutically acceptable salt thereof.

Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit.

### Definitions

As used herein, the term “about” means “approximately” (e.g., plus or minus approximately 10% of the indicated value).

As used herein, the term “compound” as used herein is meant to include all stereoisomers, geometric isomers, tautomers, and isotopes of the structures named or depicted. Compounds herein identified by name or structure as one particular tautomeric form are intended to include other tautomeric forms unless otherwise specified.

The terms “pharmaceutical” and “pharmaceutically acceptable” are employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. In some embodiments, the cell is a mesenchymal cell. In some embodiments, the cell is a fibroblast (e.g., cardiac, dermal or lung fibroblast). In some embodiments, the cell is a hepatic stellate cell.

As used herein, the term “contacting” refers to the bringing together of indicated moieties or items in an in vitro system, an ex vivo system, or an in vivo system. For example, “contacting” a cell with a compound provided herein includes the act of administering that compound to a mammal (e.g., a human) containing that cell as well as, for example, introducing that compound into a cell culture containing that cell.

As used herein, the term “mammal” includes, without limitation, mice, rats, other rodents, rabbits, dogs, cats, swine, cattle, sheep, horses, elephants, deer, non-human primates (e.g., monkeys and apes), house pets, and humans.

As used herein, the phrase “effective amount” or “therapeutically effective amount” refers to the amount of active compound or pharmaceutical agent that elicits the biological or medicinal response in a tissue, system, mammal, or human that is being sought by a researcher, veterinarian, medical doctor, or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “pharmaceutically acceptable salt” refers to a salt that is formed between an acid and a basic group of the compound, such as an amino functional group, or a base and an acidic group of the compound, such as a carboxyl functional group. In some embodiments, the compound is a pharmaceutically acceptable acid addition salt. In some embodiments, acids commonly employed to form pharmaceutically acceptable salts of the therapeutic compounds described herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the therapeutic compounds described herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

As used herein, the phrase “optionally substituted” means unsubstituted or substituted. The substituents are independently selected, and substitution can be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, without limitation, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘhaloalkyl”, employed alone or in combination with other terms, refers to an alkyl group having from one halogen atom to 2s+1 halogen atoms that may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Suitable examples of alkylamino groups include N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di Cₙ₋ₘ alkylamino” refers to a group of formula —N(alkyl)₂, wherein each alkyl group independently has n to m carbon atoms. In some embodiments, each alkyl group has 1 to 6, 1 to 4, or 1 to 3 carbon atoms. Suitable examples of dialkylamino groups include N,N-methylehtylamino, N,N-diethylamino, N,N-propylethylamino, N,N-butylisopropylamino, and the like.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “HO—C₁₋₃ alkyl” refers to a group of formula —(C₁₋₃ alkylene)-OH.

As used herein, the term “NH₂—C₁₋₃ alkyl” refers to a group of formula —(C₁₋₃ alkylene)-NH₂.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘaryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur, and oxygen. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from nitrogen, sulfur, and oxygen. In some embodiments, the heteroaryl is a 5-6 monocyclic heteroaryl having 1 or 2 heteroatom ring members independently selected from nitrogen, sulfur, and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls include, without limitation, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls include, without limitation, pyridyl, pyrazinyl, pyrimidinyl, triazinyl, and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9-, or 10-membered heterocycloalkyl groups. Heterocycloalkyl groups can also include spirocycles. Example heterocycloalkyl groups include, without limitation, pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

### EXAMPLES

Materials and Methods

Cell culture: ARPE-19 cells were purchased from ATCC (Manassas, VA, USA) were cultured in Eagle's minimal essential medium (EMEM) containing 10% fetal bovine serum (FBS) and antibiotic-antimycotic (Thermo Fisher Scientific) unless otherwise noted.

qPCR analysis: ARPE-19 cells were plated on tissue culture dishes (Thermo Fisher Scientific, 60×15 mm) in EMEM containing 10% FBS and allowed to attach and grow for 24 h. Cells were stimulated with 5 ng/ml TGF-β1 and 10 μM MS-9 or 1 μM CTC-6 in EMEM containing 0% FBS for 24 h prior to RNA isolation using the RNeasy Plus Mini Kit (Qiagen, Hilden, Germany) according to the manufacturer's instructions. Isolated RNA (500 ng) was then used to synthesize cDNA using SuperScript VILO (Invitrogen, Carlsbad, CA, USA). Quantitative PCR (qPCR) was performed using FastStart Essential DNA Green Master and analyzed with LightCycler 96 (Roche, Basel, Switzerland). Data are expressed as a fold change by ΔΔCt relative to the level of the GAPDH housekeeping gene, and normalized to control.

### Example 1—Inhibition of EMT in RPE Cells

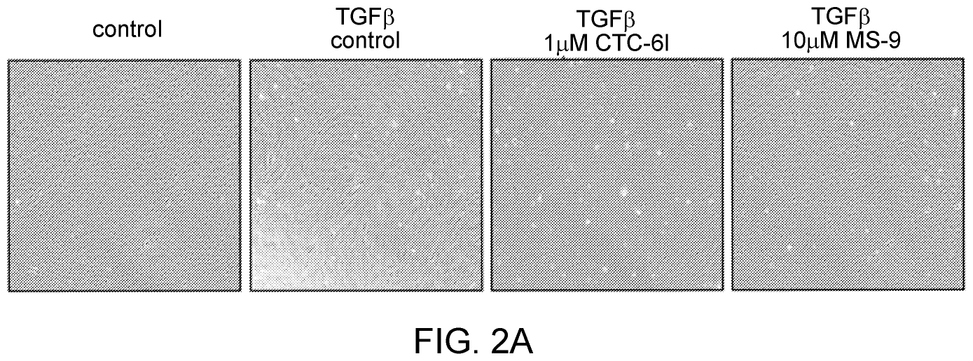

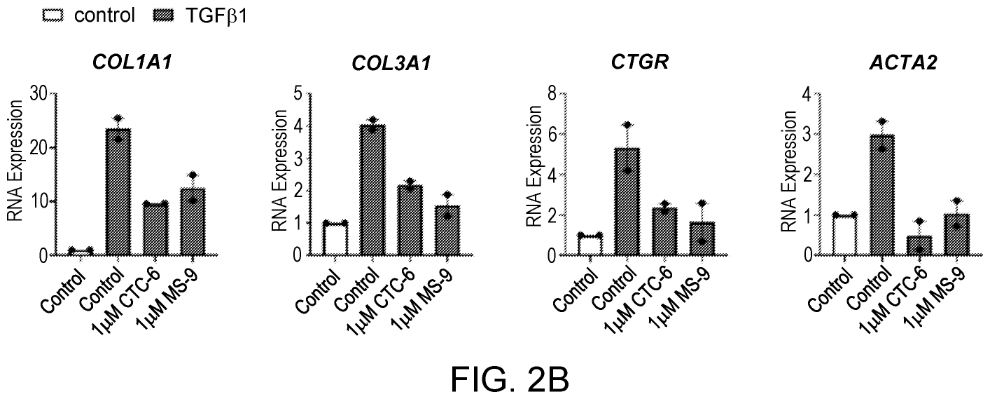

EMT was experimentally induced in cultured RPE cells by treating them with TGFβ for 24 hours and the D1-like family agonists (agonists of the D5 dopamine receptor) CTC-6 and MS-9, prior to collecting RNA to measure changes in mesenchymal associated/profibrotic genes. As expected, TGFβ stimulated morphological changes consistent with EMT (FIG. 2A) and enhanced expression of ECM genes: COL1A1 and COL3A1, connective tissue growth factor (CTGF) and alpha-smooth muscle actin (ACTA2) (FIG. 2B). Treatment with the D1 family agonists (D5 agonists) CTC-6 or MS-9 blocked markers of EMT.

### Example 2—D1 and/or D5 Dopamine Receptor Agonists Inhibit Profibrotic Gene Expression, Migration, Proliferation, and Fibronectin Deposition and Thus May Serve as Effective Mechanisms for Treating Retinal Fibrosis

Cell culture: The human RPE cell line ARPE-19 was purchased from ATCC and cultured in Dulbecco's Modified Eagle Medium/Nutrient Mixture F-12 (DMEM/F-12) supplemented with 2.50 mM L-Glutamine, 15 mM HEPES Buffer, 10% fetal bovine serum (FBS), and 1% Antibiotic-Antimycotic (Gibco), unless otherwise noted. Cells were maintained in a humidified 37° C., 5% CO₂ incubator. All experiments were performed with cells at passage 3-6.

Chemicals and Reagents: dimethyl sulfoxide (DMSO) was purchased from Fisher Scientific and used to solubilize fenoldopam and SCH 39166. 2-mercaptoethanol was purchased from Bio-Rad Laboratories and added to RLT buffer prior to RNA isolation, as per Qiagen instructions. Fenoldopam and SCH 39166 were purchased from Cayman Chemical Company. TGFβ was purchased from InvivoGen. Fenoldopam, like a majority of D5 agonists, has a characteristically short half-life. To take this into account we changed media daily to assess the efficacy of fenoldopam to inhibit fibrotic activation in vitro.

RNA Isolation/qPCR Analysis: ARPE-19 cells were plated into 12 well plates (100,000 cells/well) and allowed to attach. Media was removed and replaced with the media specified in the “Cell Culture” section above, minus FBS. Cells were treated for 24 hours ±TGFβ (10 ng/mL), 10 μM fenoldopam, and 3 μM SCH 39166. RNA was isolated using the RNeasy Plus Mini Kit (Qiagen) and the manufacturer's protocol. Isolated RNA was converted to cDNA using the SuperScript VILO cDNA Synthesis Kit (Invitrogen) and the PTC-200 Peltier Thermal Cycler (MJ Research). Quantitative PCR (qPCR) was performed using FastStart Essential DNA Green Master (Roche) and LightCycler 96 (Roche). Data are expressed as a fold change by ΔΔCt relative to GAPDH. qPCR primers (IDT) are shown in Table 1.

TABLE 1
Primers used in qPCR Analysis

| | Sequence |
|---|---|
| GAPDH | Forward: GGAAGGGCTCATGACCACAG |
| | Reverse: ACAGTCTTCTGGGTGGCAGTG |
| COL1A1 | Forward: AAGGGACACAGAGGTTTCAGT GG |
| | Reverse: CAGCACCAGTAGCACCATCATTTC |
| ACTA2 | Forward: GTGAAGAAGAGGACAGCACTG |
| | Reverse: CCCATTCCCACCATC ACC |
| CTGF | Forward: GTCCAGCACGAGGCTCA |
| | Reverse: TCGCCTTCGTGGTCCTC |
| FN1 | Forward: TGTCAGTCAAAGCAAGCCCG |
| | Reverse: TTAGGACGCTCATAAGTGTCACCC |

Proliferation and Live/Dead Assays: ARPE-19 cells were plated into 96-well plates (1,000 cells/well) and allowed to attach. Media was removed and replaced with the media specified in the “Cell Culture” section above, with reduced FBS. Cells were treated for four days ±2% FBS, and 10 μM fenoldopam, replacing media conditions every 24 hours for wells treated with fenoldopam. After four days, CellTiter-Fluor™ (Promega) was added to each well according to the manufacturer and measured on a Flexstation 3 (Molecular Devices) plate reader for the proliferation assay. For the LIVE/DEAD™ assay, each component of the kit (Thermo Fisher Scientific) was added, then cells were incubated for 30 minutes. Fluorescent images were then taken using a Cytation 5 imaging reader (BioTek) and quantified for cytotoxicity/viability.

Fibronectin Deposition: ARPE-19 cells were plated into 96-well plates (10,000 cells/well) and allowed to attach. Media was removed and replaced with the media specified in the “Cell Culture” section above, without FBS. Cells were treated for four days ±TGFβ (10 ng/mL), 10 μM fenoldopam, replacing media conditions every 24 hours for wells treated with fenoldopam. Cells were fixed with 10% neutral buffered formalin (Sigma-Aldrich) for 15 minutes. Cells were permeabilized with 0.25% Triton X-100 (Sigma-Aldrich) and blocked with 1% BSA for 1 hour. Cells were incubated with mouse monoclonal IgG₃ primary antibodies against fibronectin (Santa Cruz Biotechnology), which were diluted 1:400 in 0.25% Triton X-100 (Sigma-Aldrich) with 1% BSA. Cells were incubated with primary antibodies either in 4° C. overnight or at room temperature for 2 hours. Cells were washed with PBS (Gibco), then incubated for 1 hour with Alexa Fluor® 488 donkey anti-mouse IgG (H+L) secondary antibodies (Life Technologies) and DAPI (Thermo Fisher Scientific), which were each diluted 1:1000 in 0.25% Triton X-100 (Sigma-Aldrich) with 1% BSA. Cells were washed again with PBS (Gibco). Immunofluorescence was measured using a Cytation 5 imaging reader (BioTek).

Wound Healing Assay: ARPE-19 cells were plated confluent into a 12-well plate (300,000 cells/well) and allowed to attach. Cells were exposed to a single scratch made with a p200 pipette tip. Media was removed and replaced with the media specified in the “Cell Culture” section above, without FBS. Cells were treated ±TGFβ (10 ng/mL), 10 μM fenoldopam replacing media conditions every 24 hours for wells treated with fenoldopam. Cells were imaged after treatment for 0, 24, 48, and 72 hours. Cells were imaged with an inverted phase-contract microscope and wound area was measured using ImageJ software as previously described.

Dopamine ELISA Assay: ARPE-19 cells were plated into a 12-well plate (300,000 cells/well) and allowed to attach. Media was removed and replaced with the media specified in the “Cell Culture” section above, without FBS. Cells were treated ±TGFβ (10 ng/mL). Media was collected from cultured cells after treatment for 24 hours. Dopamine levels in the media were measured using the Mouse Dopamine ELISA kit (MyBioSource) and the manufacturer's protocol. Dopamine was quantified using the FlexStation 3 plate reader (Molecular Devices).

Statistics: in experiments comparing groups of three or more, groups were compared by one-way analysis of variance with Tukey's post-hoc comparison after confirming that data displayed a normal distribution. In experiments comparing two groups, data were compared using t-test with Welch's correction. Results are expressed throughout as the mean±standard error of the mean (SEM) with each individual datapoints shown. Statistical tests were carried out using GraphPad Prism 9 with statistical significance defined in each figure legend.

Results

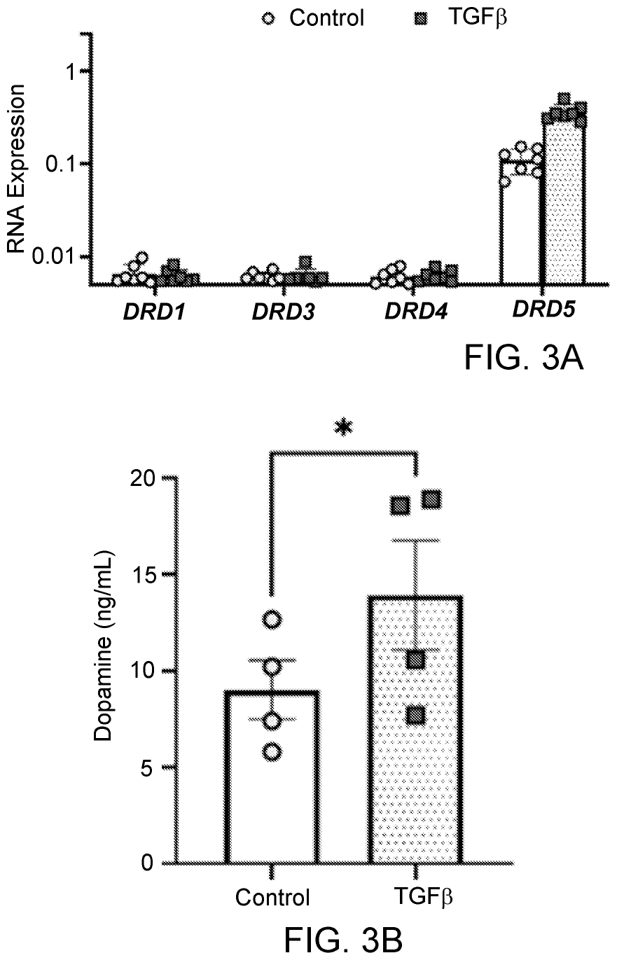

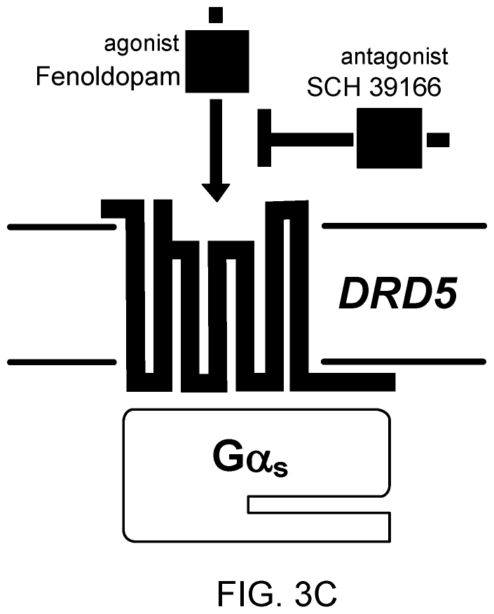

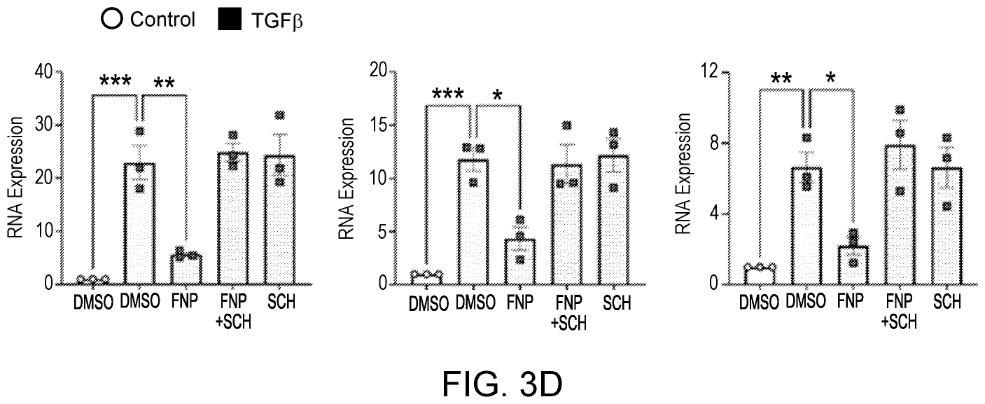

The experimental data shows that DRD5 is dominantly expressed in the cells (FIG. 3A) compared to DRD1,3,4. Moreover, DRD5 expression is elevated in the presence of TGFβ (FIG. 3A).

Expression of Gus coupled receptors like DRD5 are often repressed in response to profibrotic stimuli in cultured mesenchymal cells from other organs highlighting a potential uniqueness of the D5 receptor in this context. These findings suggest that D5 dopamine receptors are present in RPE cells and have potential to regulate their fibrotic activation. To evaluate dopamine production in RPE cells, an ELISA assay was performed on the conditioned media collected from ARPE-19 cells treated ±TGFβ for 24 hours. The data demonstrate that ARPE-19 cells produced dopamine (FIG. 3B), as was previously shown by HPLC analysis. Moreover, enhanced dopamine production in the presence of TGFβ was shown (FIG. 3B). These findings show that dopamine receptor signaling is important in regulating the fibrotic activity of RPE cells.

After identifying the preferential expression of DRD5 in ARPE-19 cells, the regulatory effects of these receptors were shown on fibrotic activation. RNA isolation and qPCR were used to measure the transcript levels of fibrotic markers in ARPE-19 cells treated ±TGFβ and tested compound. TGFβ treatment alone dramatically enhanced expression of COL1A1 (type I collagen), ACTA2 (α-smooth muscle actin), and FN1 (fibronectin), consistent with previous findings identifying TGFβ being a major contributor to epithelial-mesenchymal transition and subsequent fibrosis associated with PVR. FIGS. 3C and 3D show results for tested compounds fenoldopam (D5 agonist) and SCH 39166 (D5 antagonist). RPE cells treated with TGFβ and fenoldopam exhibited a significant decrease in the expression of COL1A1, ACTA2, and FN1 compared to those treated with TGFβ exclusively (FIG. 3D). The activity of fenoldopam being dependent on D5 dopamine receptor specifically is further supported by the data, where the D5 receptor antagonist (SCH) was able to surmount the antifibrotic effects of fenoldopam (FIG. 3D). The D5 receptor antagonist alone in the presence of TGFβ has no effect (FIG. 3D).

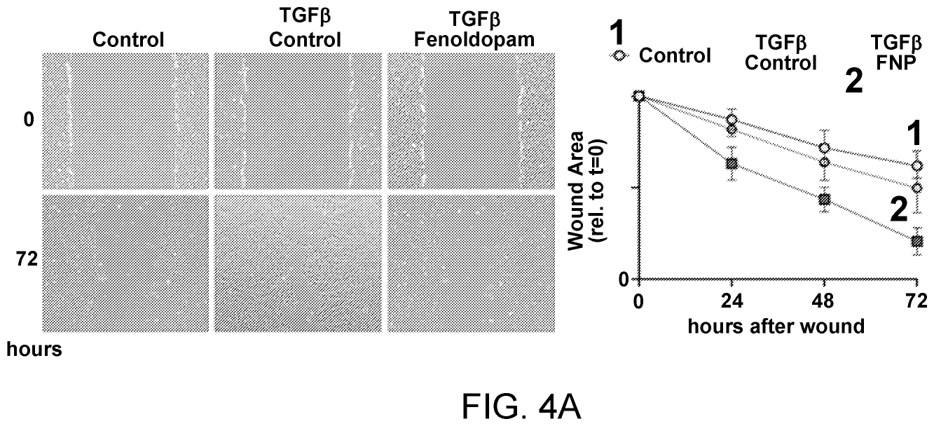

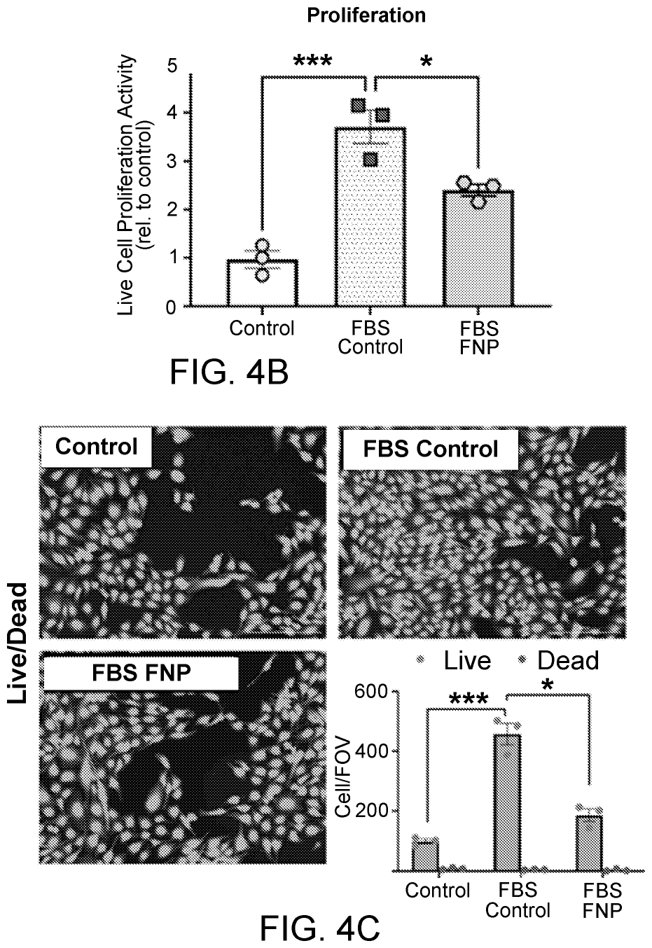

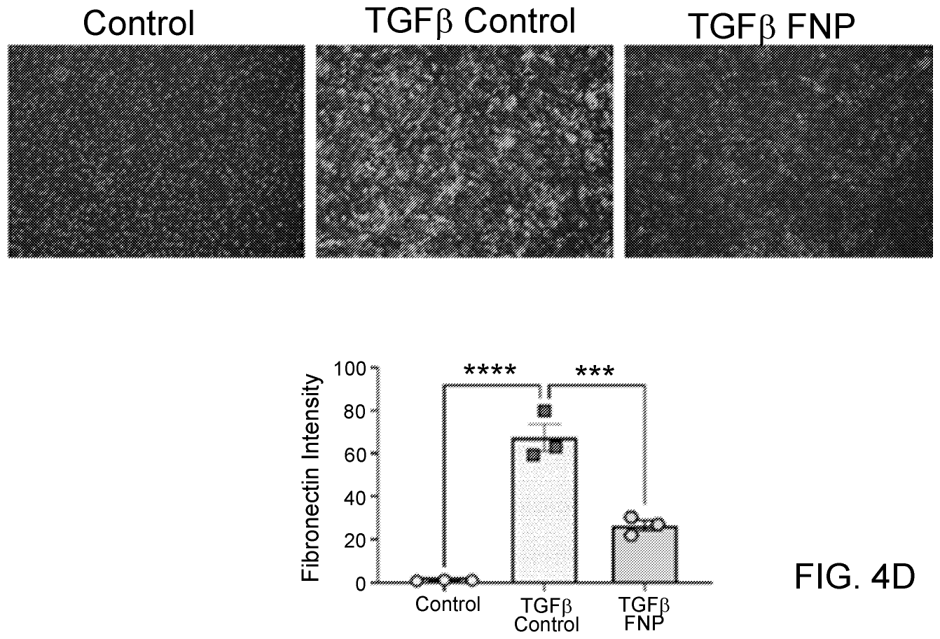

The experimental results demonstrate that agonism of the D5 receptor (fenoldopam) provides therapeutic antifibrotic effect. In vitro assays were performed to capture the pathogenic phenotypes of RPE cells in PVR: enhanced migration, aberrant proliferation, and excess ECM deposition. To confirm that D5 dopamine receptor agonism inhibits fibrotic activity in RPE cells, the wound migration, proliferation, live/dead, and fibronectin deposition assays in ARPE-19 cells treated TGFβ, or 2% FBS, and fenoldopam (FIG. 4A-4D) were performed. Fenoldopam provided a robust antifibrotic response in these assays.

### DISCUSSION

The results show that DRD5 is preferentially expressed in RPE cells. Moreover, DRD5 activation blocks profibrotic activity. DRD5 signaling has been identified as an antifibrotic regulator of cardiac fibrosis. For example, fenoldopam acts as a selective agonist of D1-like receptors, including DRD5 and is clinically approved for rare instances of postoperative emergency hypertension and is given intravenously. Further, non-catechol, chemically stable, D1-like agonists, such as those within the present claims or the numbered paragraphs, are effective in ocular fibrotic pathologies such as PVR.

### REFERENCES

1. Zhou, M., et al. Role of Epithelial-Mesenchymal Transition in Retinal Pigment Epithelium Dysfunction. Front Cell Dev Biol 8(2020).
2. Takahashi, E., et al. Tumor Necrosis Factor-alpha Regulates Transforming Growth Factor-beta-dependent Epithelial-Mesenchymal Transition by Promoting Hyaluronan-CD44-Moesin Interaction. Journal of Biological Chemistry 285, 4060-4073 (2010).
3. Beaulieu, J. M., and Gainetdinov, R. R. (2011). The Physiology, Signaling, and Pharmacology of Dopamine Receptors. Pharmacol Rev 63, 182-217.
4. Bell, J. L., Haak, A. J., Wade, S. M., Kirchhoff, P. D., Neubig, R. R., and Larsen, S. D. (2013). Optimization of novel nipecotic bis(amide) inhibitors of the Rho/MKL1/SRF transcriptional pathway as potential anti-metastasis agents. Bioorg Med Chem Lett 23, 3826-3832.
5. Boyd, K. N., and Mailman, R. B. (2012). Dopamine receptor signaling and current and future antipsychotic drugs. Handb Exp Pharmacol, 53-86.
6. Breslin, S., and O'Driscoll, L. (2013). Three-dimensional cell culture: the missing link in drug discovery. Drug Discov Today 18, 240-249.
7. Brogden, R. N., and Markham, A. (1997). Fenoldopam: a review of its pharmacodynamic and pharmacokinetic properties and intravenous clinical potential in the management of hypertensive urgencies and emergencies. Drugs 54, 634-650.
8. Choi, K. M., Haak, A. J., Diaz Espinosa, A. M., Cummins, K. A., Link, P. A., Aravamudhan, A., Wood, D. K., and Tschumperlin, D. J. (2021). GPCR-mediated YAP/TAZ inactivation in fibroblasts via EPAC1/2, RAP2C, and MAP4K7. J Cell Physiol 236, 7759-7774.
9. Diaz-Espinosa, A. M., Link, P. A., Sicard, D., Jorba, I., Tschumperlin, D. J., and Haak, A. J. (2020). Dopamine D1 receptor stimulates cathepsin K-dependent degradation and resorption of collagen I in lung fibroblasts. J Cell Sci 133.
11. Friedlander, M. (2007). Fibrosis and diseases of the eye. J Clin Invest 117, 576-586.
12. Gray, D. L., Allen, J. A., Mente, S., O'Connor, R. E., DeMarco, G. J., Efremov, I., Tierney, P., Volfson, D., Davoren, J., Guilmette, E., et al. (2018). Impaired beta-arrestin recruitment and reduced desensitization by non-catechol agonists of the D1 dopamine receptor. Nat Commun 9, 674.
13. Guha, S., Baltazar, G. C., Tu, L. A., Liu, J., Lim, J. C., Lu, W., Argall, A., Boesze-Battaglia, K., Laties, A. M., and Mitchell, C. H. (2012). Stimulation of the D5 dopamine receptor acidifies the lysosomal pH of retinal pigmented epithelial cells and decreases accumulation of autofluorescent photoreceptor debris. J Neurochem 122, 823-833.
14. Haak, A. J., Ducharme, M. T., Espinosa, A. M. D., and Tschumperlin, D. J. (2020). Targeting GPCR Signaling for Idiopathic Pulmonary Fibrosis Therapies. Trends in Pharmacological Sciences 41, 172-182.
15. Haak, A. J., Kostallari, E., Sicard, D., Ligresti, G., Choi, K. M., Caporarello, N., Jones, D. L., Tan, Q., Meridew, J., Diaz Espinosa, A. M., et al. (2019). Selective YAP/TAZ inhibition in fibroblasts via dopamine receptor D1 agonism reverses fibrosis. Sci Transl Med 11.
16. Hazim, R. A., Volland, S., Yen, A., Burgess, B. L., and Williams, D. S. (2019). Rapid differentiation of the human RPE cell line, ARPE-19, induced by nicotinamide. Exp Eye Res 179, 18-24.
17. Idrees, S., Sridhar, J., and Kuriyan, A. E. (2019). Proliferative Vitreoretinopathy: A Review. Int Ophthalmol Clin 59, 221-240.
18. Insel, P. A., Murray, F., Yokoyama, U., Romano, S., Yun, H., Brown, L., Snead, A., Lu, D., and Aroonsakool, N. (2012). cAMP and Epac in the regulation of tissue fibrosis. Br J Pharmacol 166, 447-456.
19. Jiang, X., Shao, M., Liu, X., Liu, X., Zhang, X., Wang, Y., Yin, K., Wang, S., Hu, Y., Jose, P. A., et al. (2021a). Reversible Treatment of Pressure Overload-Induced Left Ventricular Hypertrophy through Drd5 Nucleic Acid Delivery Mediated by Functional Polyaminoglycoside. Adv Sci (Weinh) 8, 2003706.
20. Jiang, X. L., Shao, M. Y., Liu, X., Liu, X., Zhang, X., Wang, Y. M., Yin, K. L., Wang, S. Y., Hu, Y., Jose, P. A., et al. (2021b). Reversible Treatment of Pressure Overload-Induced Left Ventricular Hypertrophy through Drd5 Nucleic Acid Delivery Mediated by Functional Polyaminoglycoside. Adv Sci 8.
21. Kirchhof, B., and Sorgente, N. (1989). Pathogenesis of proliferative vitreoretinopathy. Modulation of retinal pigment epithelial cell functions by vitreous and macrophages. Dev Ophthalmol 16, 1-53.
22. Kita, T., Hata, Y., Arita, R., Kawahara, S., Miura, M., Nakao, S., Mochizuki, Y., Enaida, H., Goto, Y., Shimokawa, H., et al. (2008). Role of TGF-beta in proliferative vitreoretinal diseases and ROCK as a therapeutic target. P Natl Acad Sci USA 105, 17504-17509.
23. Martucci, A., Cesareo, M., Pinazo-Duran, M. D., Di Pierro, M., Di Marino, M., Nucci, C., Coletta, M., and Mancino, R. (2020). Is there a relationship between dopamine and rhegmatogenous retinal detachment? Neural Regen Res 15, 311-314.
24. Miller, C. G., Budoff, G., Prenner, J. L., and Schwarzbauer, J. E. (2017). Minireview: Fibronectin in retinal disease. Exp Biol Med (Maywood) 242, 1-7.
25. Ming, M., Li, X. P., Fan, X. L., Yang, D. H., Li, L., Chen, S., Gu, Q., and Le, W. D. (2009). Retinal pigment epithelial cells secrete neurotrophic factors and synthesize dopamine: possible contribution to therapeutic effects of RPE cell transplantation in Parkinson's disease. J Transl Med 7.
26. Mou, Y., Liu, J., Pan, T., Wang, Q., Miao, K., Xu, Y., Xiong, W., and Yu, J. (2021). Dopamine receptor agonists ameliorate bleomycin-induced pulmonary fibrosis by repressing fibroblast differentiation and proliferation. Biomed Pharmacother 139, 111500.
27. Mudhar, H. S. (2020). A brief review of the histopathology of proliferative vitreoretinopathy (PVR). Eye (Lond) 34, 246-250.
28. Orita, T., Kimura, K., and Sonoda, K. (2013). Effect of prostaglandin E2 on collagen gel contraction in mouse retinal pigment epithelium cells. Invest Ophth Vis Sci 54.
31. Roy, S., Amin, S., and Roy, S. (2016). Retinal fibrosis in diabetic retinopathy. Exp Eye Res 142, 71-75.
32. Seagle, B. L. L., Rezai, K. A., Kobori, Y., Gasyna, E. M., Rezaei, K. A., and Norris, J. R. (2005). Melanin photoprotection in the human retinal pigment epithelium and its correlation with light-induced cell apoptosis. P Natl Acad Sci USA 102, 8978-8983.
33. Sparrow, J. R., Hicks, D., and Hamel, C. P. (2010). The retinal pigment epithelium in health and disease. Curr Mol Med 10, 802-823.
34. Sriram, K., and Insel, P. A. (2018). G Protein-Coupled Receptors as Targets for Approved Drugs: How Many Targets and How Many Drugs? Mol Pharmacol 93, 251-258.
35. Tamiya, S., and Kaplan, H. J. (2016). Role of epithelial-mesenchymal transition in proliferative vitreoretinopathy. Exp Eye Res 142, 26-31.
36. Yin, F., Tian, Z. M., Liu, S., Zhao, Q. J., Wang, R. M., Shen, L., Wieman, J., and Yan, Y. (2012). Transplantation of Human Retinal Pigment Epithelium Cells in the Treatment for Parkinson Disease. Cns Neurosci Ther 18, 1012-1020.

### NUMBERED PARAGRAPHS

Paragraph 1. A method of treating or preventing an ocular fibrotic pathology, the method comprising administering to a subject in need thereof a therapeutically effective amount of a dopamine receptor agonist, or a pharmaceutically acceptable salt thereof.
Paragraph 2. The method of paragraph 1, wherein the dopamine receptor is dopamine receptor D1 (DRD1).
Paragraph 3. The method of paragraph 1, wherein the dopamine receptor is dopamine receptor D5 (DRD5).
Paragraph 4. The method of any one of paragraphs 1-3, wherein the ocular fibrotic pathology is selected from: proliferative vitreoretinopathy (PVR), diabetic retinopathy, ischemic retinopathy, age-related macular degeneration (ARMD), dry ARMD, neovascular ARMD, keratitis, pterygia, pingueculae, retinopathy of prematurity, glaucoma (including neovascular glaucoma, open-angle glaucoma, angle-closure glaucoma, secondary glaucoma, and childhood glaucoma), Stargardt's disease, sickle cell retinopathy, radiation retinopathy, optic neuropathy, retinal detachment, retinal degeneration, uveitis, dry eye disease, congenital fibrosis of the extraocular muscles (CFEOM), and comeal fibrosis.
Paragraph 5. The method of paragraph 4, wherein the ocular fibrotic pathology is proliferative vitreoretinopathy (“PVR”).
Paragraph 6. The method of any one of paragraphs 1-3, wherein the ocular fibrotic pathology is selected from: opacification and fibrosis of the posterior capsule of the lens following eye surgery, fibrosis following glaucoma filtration surgery, fibrosis following a wound or trauma, conjunctival fibrosis or subconjunctival fibrosis, fibrosis of the ocular muscles, Graves disease, fibrosis following wound healing of the skin around the eye and face, fibrosis of the surface of the eye with pterygium or pingueculae, fibrosis due to choroidal neovascularization and angiogenesis, fibrosis following a corneal wound, fibrosis following corneal laser surgery, fibrosis following refractive surgery, and fibrosis following a corneal transplant.
Paragraph 7. The method of any one of paragraphs 1-6, wherein the dopamine receptor agonist is a compound of Formula (I):

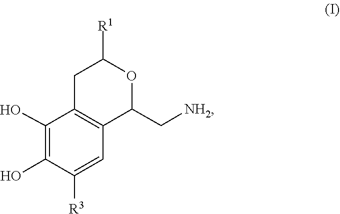

or a pharmaceutically acceptable salt thereof, wherein:
R¹ is selected from HO—C₁₋₆ alkyl, NH₂-C₁₋₆ alkyl, C₆₋₁₂ aryl ring, 5-6-membered heteroaryl ring comprising 1 to 5 heteroatoms selected from N, O, and S, and 3-10-membered heterocycloalkyl ring comprising 1 to 3 heteroatoms independently selected from N, O, and S;
wherein said heteroaryl ring and heterocycloalkyl ring are each optionally substituted with 1, 2, or 3 substituents independently selected from R²;
each R² is independently selected from halo, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino; and
R³ is selected from H and halo.
Paragraph 8. The method of paragraph 7, wherein the compound of Formula (I) has formula

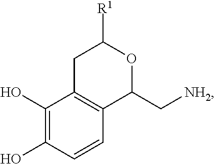

or a pharmaceutically acceptable salt thereof.
Paragraph 9. The method of paragraph 7, wherein R¹ is C₆₋₁₂ aryl ring, which is optionally substituted with 1, 2, or 3 substituents independently selected from R².
Paragraph 10. The method of paragraph 7, wherein the compound of Formula (I) has formula:

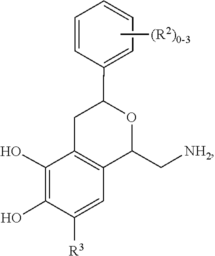

or a pharmaceutically acceptable salt thereof.
Paragraph 11. The method of paragraph 7, wherein R¹ is 5-6-membered heteroaryl ring comprising 1 to 5 heteroatoms selected from N, O, and S, which is optionally substituted with 1, 2, or 3 substituents independently selected from R.
Paragraph 12. The method of paragraph 7, wherein R¹ is 5-6-membered heteroaryl ring comprising 1 or 2 heteroatoms selected from N, O, and S, which is optionally substituted with 1, 2, or 3 substituents independently selected from R.
Paragraph 13. The method of paragraph 7, wherein R¹ is selected from pyridinyl, pyrimidinyl, pyrazinyl, diazinyl, triazinyl, tetrazinyl, and pentazinyl, each of which is optionally substituted with 1, 2, or 3 substituents independently selected from R².
Paragraph 14. The method of paragraph 7, wherein R¹ is selected from pyridinyl, pyrimidinyl, and pyrazinyl, each of which is optionally substituted with 1, 2, or 3 substituents independently selected from R.
Paragraph 15. The method of paragraph 7, wherein R¹ is pyridinyl, optionally substituted with 1, 2, or 3 substituents independently selected from R.
Paragraph 16. The method of paragraph 7, wherein the compound of Formula (I) is selected from:

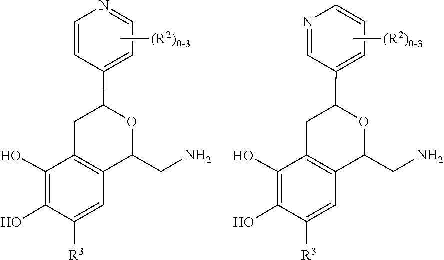

or a pharmaceutically acceptable salt thereof.
Paragraph 17. The method of paragraph 7, wherein the compound of Formula (I) is selected from:

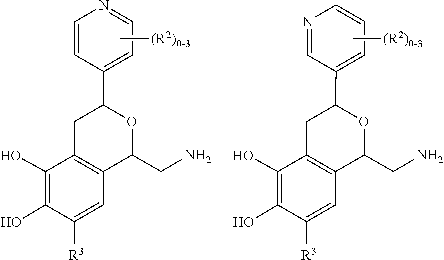

or a pharmaceutically acceptable salt thereof.
Paragraph 18. The method of paragraph 7, wherein R¹ is 3-10-membered heterocycloalkyl ring comprising 1 to 3 heteroatoms independently selected from N, O, and S, which is optionally substituted with 1, 2, or 3 substituents independently selected from R².
Paragraph 19. The method of paragraph 7, wherein R¹ is 3-7-membered heterocycloalkyl ring comprising 1 or 2 heteroatoms selected from N, O, and S, which is optionally substituted with 1, 2, or 3 substituents independently selected from R².
Paragraph 20. The method of paragraph 7, wherein R¹ is selected from tetrahydropyranyl, piperidinyl, tetrahydrofuranyl, pyrrolidinyl, pyranyl, morpholinyl, oxazinyl, dioxanyl, dioxinyl, diazinanyl, triazinanyl, trioxanyl, azepanyl, azepinyl, oxepanyl, oxepinyl, diazepanyl, diazepinyl, azocanyl, azocinyl, oxocanyl, oxocinyl, azonanyl, azoninyl, oxonanyl, and oxoninyl, each of which is optionally substituted with 1, 2, or 3 substituents independently selected from R².
Paragraph 21. The method of paragraph 7, wherein R¹ is selected from tetrahydropyranyl, piperidinyl, tetrahydrofuranyl, and pyrrolidinyl, each of which is optionally substituted with 1, 2, or 3 substituents independently selected from R².
Paragraph 22. The method of paragraph 7, wherein R¹ is tetrahydropyranyl, optionally substituted with 1, 2, or 3 substituents independently selected from R².
Paragraph 23. The method of paragraph 7, wherein the compound of Formula (I) is selected from:

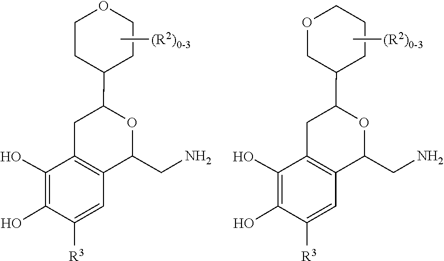

or a pharmaceutically acceptable salt thereof.
Paragraph 24. The method of paragraph 7, wherein the compound of Formula (I) is selected from:

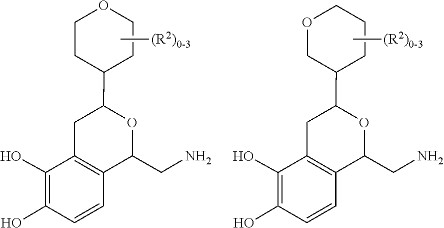

or a pharmaceutically acceptable salt thereof.
Paragraph 25. The method of paragraph 7, wherein the compound of Formula (I) is selected from:

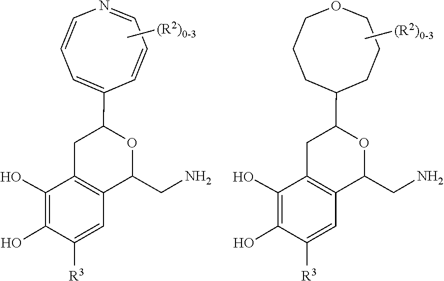

or a pharmaceutically acceptable salt thereof.
Paragraph 26. The method of paragraph 7, wherein R¹ is selected from HO—C₁₋₆ alkyl and NH₂—C₁₋₆ alkyl.
Paragraph 27. The method of paragraph 7, wherein R¹ is HO—C₁₋₆ alkyl.
Paragraph 28. The method of any one of paragraphs 7-27, wherein each R² is independently selected from halo, OH, C₁₋₃ alkoxy, C₁₋₃ alkyl, and C₁₋₃ haloalkyl.
Paragraph 29. The method of any one of paragraphs 7-27, wherein each R² is independently selected from OH, NH₂, CH₃ alkyl, HO-C₁₋₃ alkyl, and NH₂—C₁₋₃ alkyl.
Paragraph 30. The method of any one of paragraphs 7-29, wherein R³ is H.
Paragraph 31. The method of any one of paragraphs 7-29, wherein R³ is selected from Cl, F, and Br.
Paragraph 32. The method of paragraph 7, wherein the compound of Formula (I) is selected from any one of the following compounds:

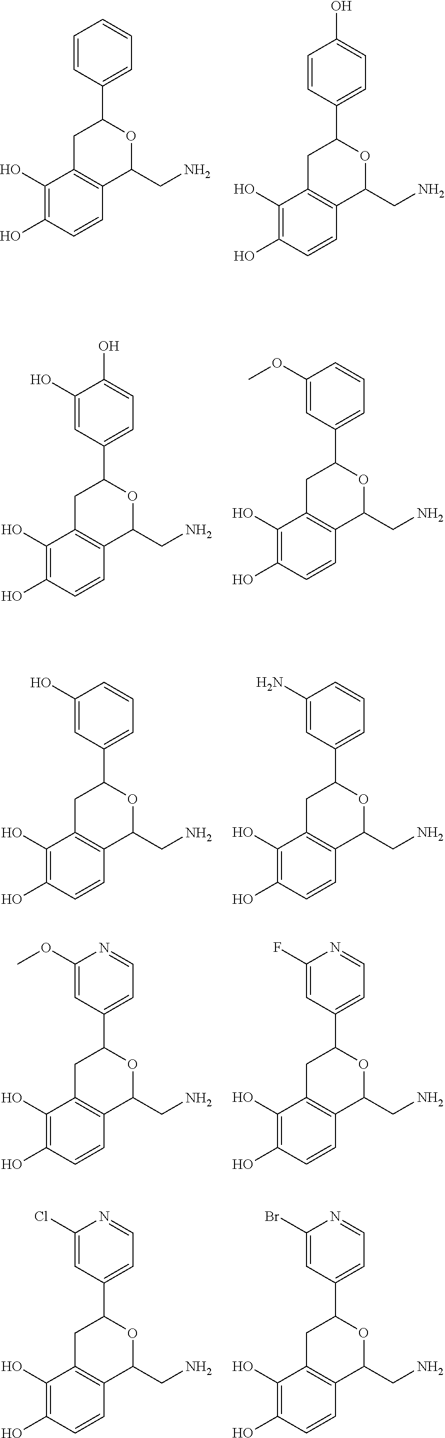

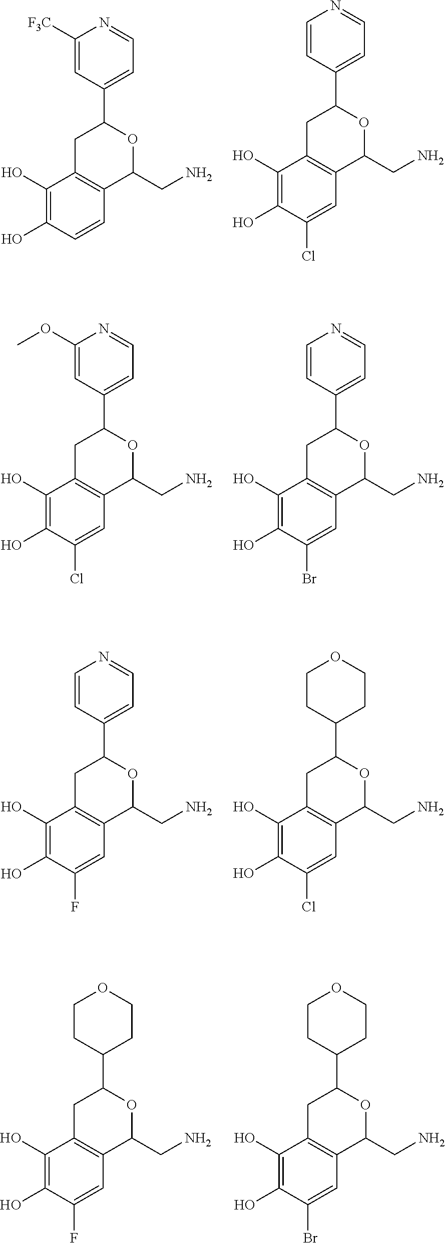

or a pharmaceutically acceptable salt thereof
Paragraph 33. The method of paragraph 7, wherein the compound of Formula (I) is selected from any one of the following compounds:

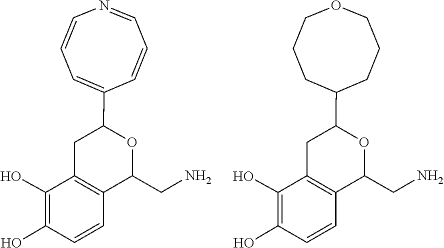 [SMILES: NCC1OC(C2=C/C=C\N=C/C=C\2)Cc2c1ccc(O)c2O ; NCC1OC(C2CCCOCCC2)Cc2c1ccc(O)c2O]

or a pharmaceutically acceptable salt thereof
Paragraph 34. The method of paragraph 7, wherein the compound of Formula (I) is selected from any one of the following compounds:

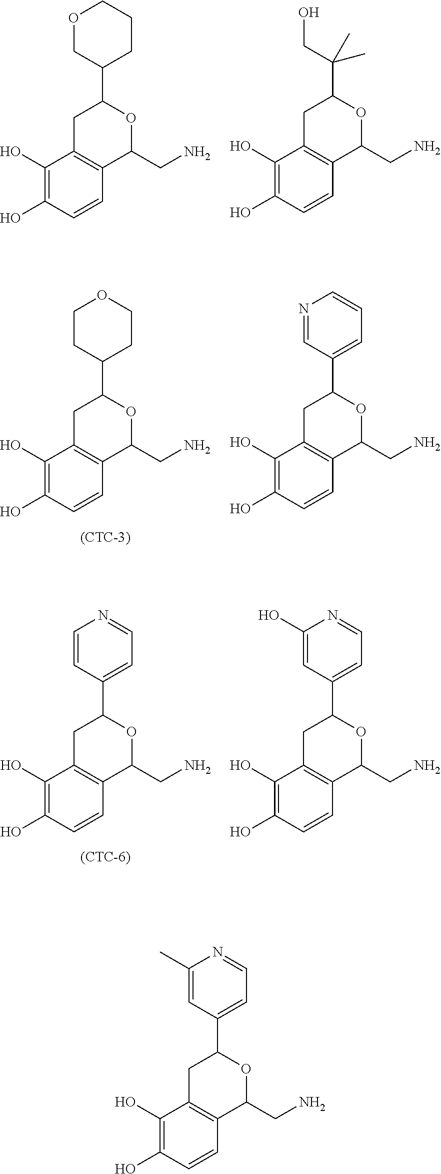

or a pharmaceutically acceptable salt thereof.
Paragraph 35. The method of any one of paragraph 1-6, wherein the dopamine receptor agonist is a compound of Formula (II):

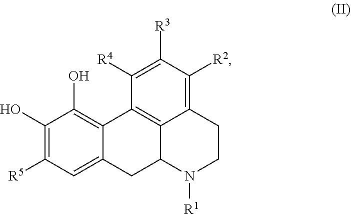

or a pharmaceutically acceptable salt thereof, wherein:
R¹ is selected from H and C₁₋₃ alkyl, wherein said C₁₋₃ alkyl is optionally substituted with OH, SH, NH₂, C₁₋₃ alkylamino, or di(C₁₋₃ alkyl)amino;
R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino; and
R⁵ is selected from H and halo.
Paragraph 36. The method of paragraph 35, wherein the compound of Formula (II) has formula:

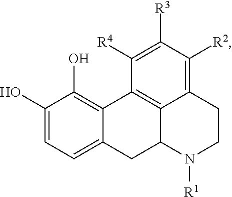

or a pharmaceutically acceptable salt thereof.
Paragraph 37. The method of paragraph 35, wherein R¹ is H.
Paragraph 38. The method of paragraph 35, wherein R¹ is C₁₋₃ alkyl.
Paragraph 39. The method of paragraph 35, wherein R¹ is selected from HO—C₁₋₃ alkyl and NH₂—C₁₋₃ alkyl.
Paragraph 40. The method of paragraph 35, wherein at least one of R², R³, and R⁴ is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.
Paragraph 41. The method of paragraph 35, wherein at least one of R², R³, and R⁴ is selected from NH₂, C₁₋₃ alkyl, HO—C₁₋₃ alkyl, and NH₂—C₁₋₃ alkyl.
Paragraph 42. The method of paragraph 35, wherein at least one of R², R³, and R⁴ is C₁₋₃ alkyl.
Paragraph 43. The method of paragraph 35, wherein:
   R³ is OH; and
   R² is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.
Paragraph 44. The method of paragraph 35, wherein:
   R³ is OH; and
   R² is selected from OH, NH₂, C₁₋₃ alkyl, HO—C₁₋₃ alkyl, and NH₂—C₁₋₃ alkyl.
Paragraph 45. The method of paragraph 35, wherein:
   R³ is OH; and
   R⁴ is selected from SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.
Paragraph 46. The method of paragraph 35, wherein:
R³ is OH; and
R⁴ is selected from NH₂, C₁₋₃ alkyl, HO—C₁₋₃ alkyl, and NH₂—C₁₋₃ alkyl.
Paragraph 47. The method of paragraph 35, wherein:
R⁴ is OH; and
R³ is selected from H, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.
Paragraph 48. The method of paragraph 35, wherein:
   R⁴ is OH; and
   R³ is selected from H, NH₂, C₁₋₃ alkyl, HO—C₁₋₃ alkyl, and NH₂—C₁₋₃ alkyl.
Paragraph 49. The method of paragraph 35, wherein:
   R² is OH; and
   at least one of R³ and R⁴ is selected from OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino and di(C₁₋₃ alkyl)amino.
Paragraph 50. The method of paragraph 35, wherein:
   R² is OH; and
   at least one of R³ and R⁴ is selected from OH, NH₂, C₁₋₃ alkyl, HO—C₁₋₃ alkyl, and NH₂—C₁₋₃ alkyl.
Paragraph 51. The method of paragraph 35, wherein:
   R⁵ is halo; and
   R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, SH, NH₂, C₁₋₃ alkylamino, and di(C₁₋₃ alkyl)amino.
Paragraph 52. The method of paragraph 35, wherein R², R³, and R⁴ are each independently selected from H, OH, and C₁₋₃ alkyl.
Paragraph 53. The method of paragraph 35, wherein R², R³, and R⁴ are each H.
Paragraph 54. The method of paragraph 35, wherein R⁵ is H.
Paragraph 55. The method of paragraph 35, wherein R⁵ is selected from Cl, Br, and F.
Paragraph 56. The method of paragraph 35, wherein the compound of Formula (II) is:

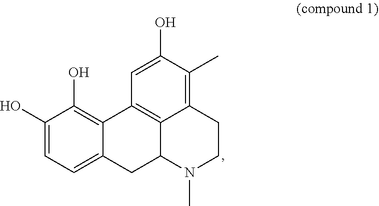 [SMILES: Cc1c(O)cc2c3c1CCN(C)C3Cc1ccc(O)c(O)c1-2]

or a pharmaceutically acceptable salt thereof.
Paragraph 57. The method of paragraph 35, wherein the compound of Formula (II) is:

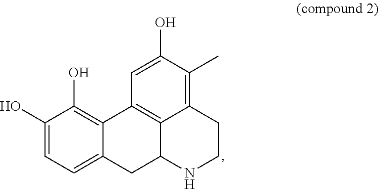 [SMILES: Cc1c(O)cc2c3c1CCNC3Cc1ccc(O)c(O)c1-2]

or a pharmaceutically acceptable salt thereof.
Paragraph 58. The method of paragraph 35, wherein the compound of Formula (II) is selected from any one of the following compounds:

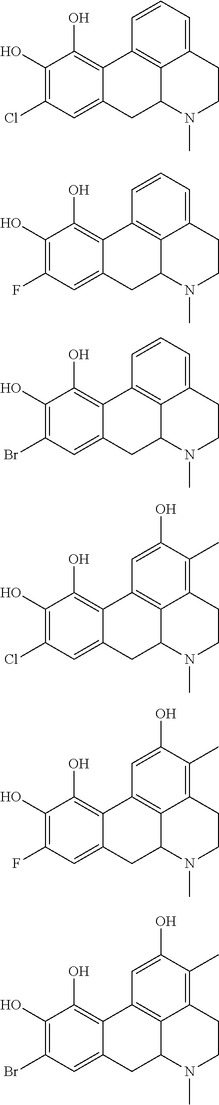

or a pharmaceutically acceptable salt thereof.
Paragraph 59. The method of any one of paragraphs 1-6, wherein the dopamine receptor agonist is a compound of Formula (III):

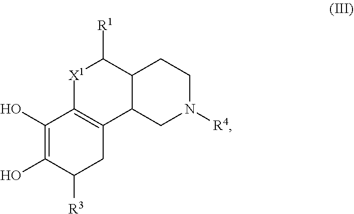

or a pharmaceutically acceptable salt thereof, wherein:
   X¹ is selected from CH₂ and O;
   R¹ is selected from HO—C₁₋₆ alkyl, NH₂—C₁₋₆ alkyl, C₆₋₁₂ aryl ring, 5-6-membered heteroaryl ring comprising 1 to 5 heteroatoms selected from N, O, and S, and 3-10-membered heterocycloalkyl ring comprising 1 to 3 heteroatoms independently selected from N, O, and S;
   wherein said heteroaryl ring and heterocycloalkyl ring are each optionally substituted with 1, 2, or 3 substituents independently selected from R²;
      each R² is independently selected from halo, OH, C₁₋₃ alkoxy, SH, NH₂, C₁₋₃ alkylamino, di(C₁₋₃ alkyl)amino, C₁₋₃ alkyl, and C₁₋₃ haloalkyl, wherein said C₁₋₃ alkyl is optionally substituted with OH, C₁₋₃ alkoxy, SH, NH₂, C₁₋₃ alkylamino, and di(C₁₋₃ alkyl)amino;
   R³ is selected from H and halo; and
   R⁴ is selected from H and C₁₋₃ alkyl, wherein said C₁₋₃ alkyl is optionally substituted with OH, SH, NH₂, C₁₋₃ alkylamino, or di(C₁₋₃ alkyl)amino.
Paragraph 60. The method of paragraph 59, wherein X¹ is CH₂.
Paragraph 61. The method of paragraph 59, wherein X¹ is O.
Paragraph 62. The method of paragraph 59, wherein R³ is H.
Paragraph 63. The method of paragraph 59, wherein R³ is halo.
Paragraph 64. The method of paragraph 59, wherein R⁴ is selected from H and C₁₋₃ alkyl.
Paragraph 65. The method of paragraph 59, wherein R⁴ is H.
Paragraph 66. The method of paragraph 59, wherein R⁴ is C₁₋₃ alkyl.
Paragraph 67. The method of paragraph 59, wherein R¹ is C₆₋₁₂ aryl ring, which is optionally substituted with 1, 2, or 3 substituents independently selected from R².
Paragraph 68. The method of paragraph 59, wherein the compound of Formula (III) has formula:

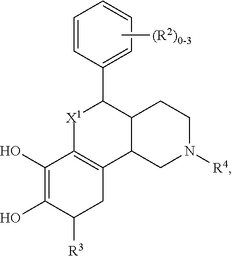

or a pharmaceutically acceptable salt thereof.
Paragraph 69. The method of paragraph 59, wherein R¹ is 5-6-membered heteroaryl ring comprising 1 to 5 heteroatoms selected from N, O, and S, which is optionally substituted with 1, 2, or 3 substituents independently selected from R.
Paragraph 70. The method of paragraph 59, wherein R¹ is selected from pyridinyl, pyrimidinyl, pyrazinyl, diazinyl, triazinyl, tetrazinyl, and pentazinyl, each of which is optionally substituted with 1, 2, or 3 substituents independently selected from R².
Paragraph 71. The method of paragraph 59, wherein R¹ is pyridinyl, optionally substituted with 1, 2, or 3 substituents independently selected from R.
Paragraph 72. The method of paragraph 59, wherein the compound of Formula (III) is selected from:

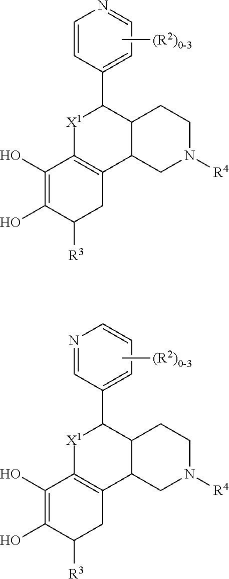

or a pharmaceutically acceptable salt thereof.
Paragraph 73. The method of paragraph 59, wherein R¹ is 3-10-membered heterocycloalkyl ring comprising 1 to 3 heteroatoms independently selected from N, O, and S, which is optionally substituted with 1, 2, or 3 substituents independently selected from R².
Paragraph 74. The method of paragraph 59, wherein R¹ is selected from tetrahydropyranyl, piperidinyl, tetrahydrofuranyl, pyrrolidinyl, pyranyl, morpholinyl, oxazinyl, dioxanyl, dioxinyl, diazinanyl, triazinanyl, trioxanyl, azepanyl, azepinyl, oxepanyl, oxepinyl, diazepanyl, diazepinyl, azocanyl, azocinyl, oxocanyl, oxocinyl, azonanyl, azoninyl, oxonanyl, and oxoninyl, each of which is optionally substituted with 1, 2, or 3 substituents independently selected from R².
Paragraph 75. The method of paragraph 59, wherein R¹ is selected from tetrahydropyranyl, piperidinyl, tetrahydrofuranyl, and pyrrolidinyl, each of which is optionally substituted with 1, 2, or 3 substituents independently selected from R².
Paragraph 76. The method of paragraph 59, wherein R¹ is tetrahydropyranyl, optionally substituted with 1, 2, or 3 substituents independently selected from R².
Paragraph The method of paragraph 59, wherein the compound of Formula (III) is selected from:

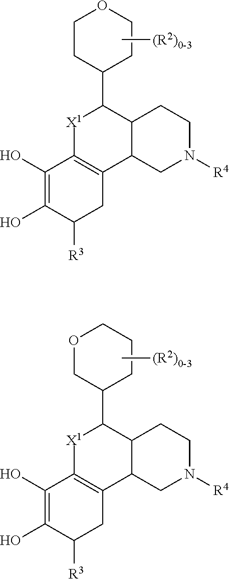

or a pharmaceutically acceptable salt thereof.
Paragraph 78. The method of paragraph 59, wherein R¹ is selected from HO—C₁₋₆ alkyl and NH₂—C₁₋₆ alkyl.
Paragraph 79. The method of paragraph 59, wherein R¹ is HO—C₁₋₆ alkyl.
Paragraph 80. The method of paragraph 59, wherein each R² is independently selected from halo, OH, C₁₋₃ alkoxy, C₁₋₃ alkyl, and C₁₋₃ haloalkyl.
Paragraph 81. The method of paragraph 59, wherein the compound of Formula (III) is selected from any one of the following compounds:

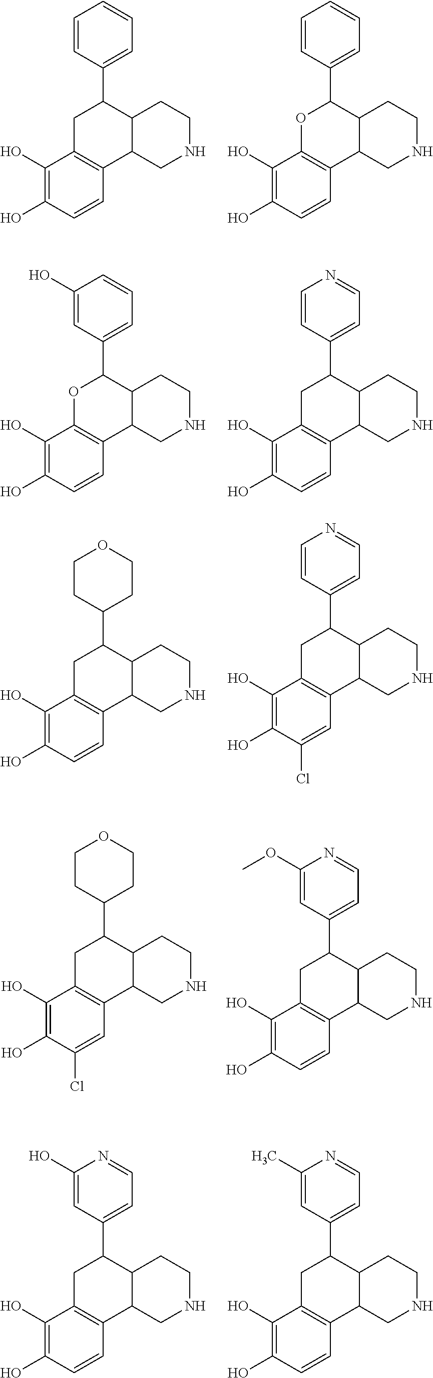

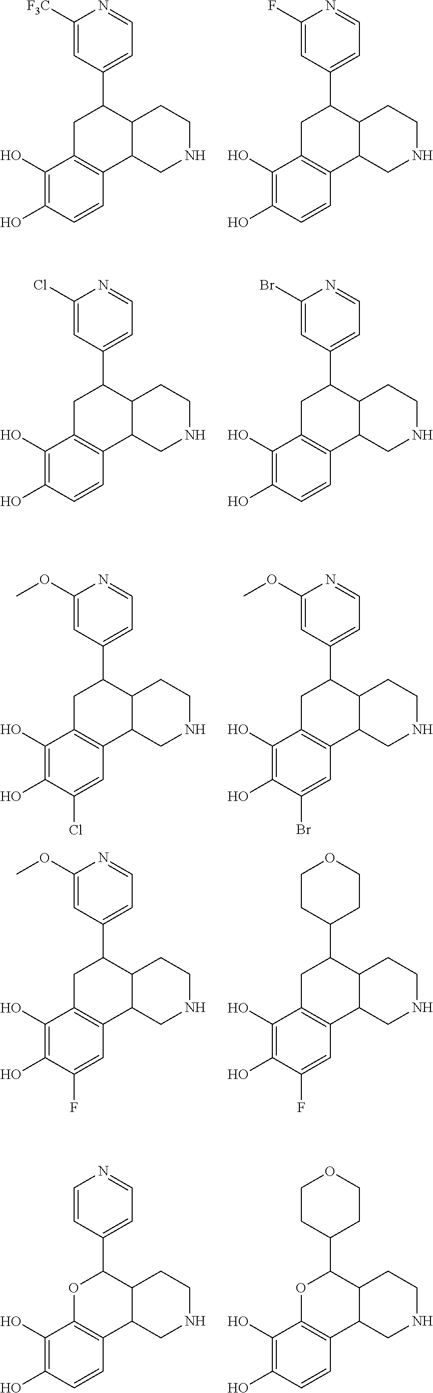

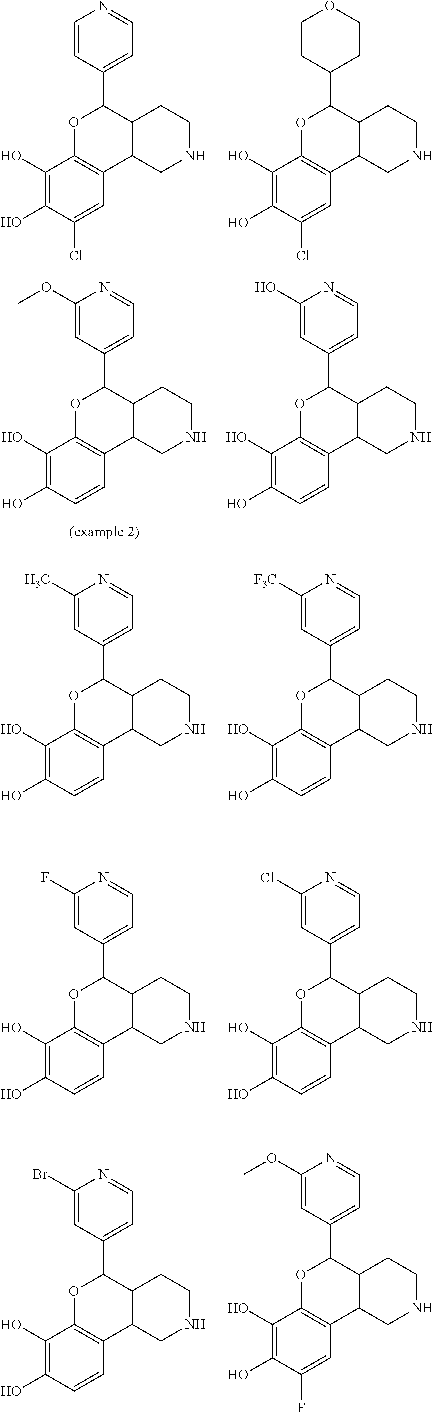

or a pharmaceutically acceptable salt thereof.
Paragraph 82. A method of any one of paragraphs 1-6, wherein the dopamine receptor agonist is a compound of Formula (IV):

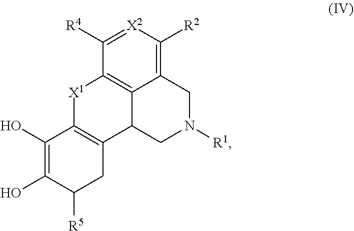

or a pharmaceutically acceptable salt thereof, wherein:
   X¹ is selected from CH₂ and O;
   X² is selected from CR³ and N;
   R¹ is selected from H and C₁₋₃ alkyl, wherein said C₁₋₃ alkyl is optionally substituted with OH, SH, NH₂, C₁₋₃ alkylamino, or di(C₁₋₃ alkyl)amino;
   R⁵ is selected from H and halo; and
   R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, SH, NH₂, C₁₋₃ alkylamino, and di(C₁₋₃ alkyl)amino.
Paragraph 83. The method of paragraph 82, wherein X¹ is CH₂.
Paragraph 84. The method of paragraph 82, wherein X¹ is O.
Paragraph 85. The method of paragraph 82, wherein R¹ is selected from H and C₁₋₃ alkyl.
Paragraph 86. The method of paragraph 82, wherein R¹ is H.
Paragraph 87. The method of paragraph 82, wherein R¹ is C₁₋₃ alkyl.
Paragraph 88. The method of paragraph 82, wherein R⁵ is H.
Paragraph 89. The method of paragraph 82, wherein R⁵ is selected from Cl, Br, and F.
Paragraph 90. The method of paragraph 82, wherein the compound of Formula (IV) has formula:

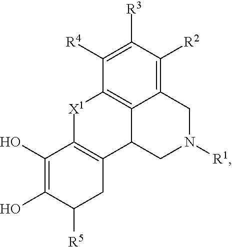

or a pharmaceutically acceptable salt thereof.
Paragraph 91. The method of paragraph 82, wherein:
   R⁵ is halo; and
   R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, SH, NH₂, C₁₋₃ alkylamino, and di(C₁₋₃ alkyl)amino.
Paragraph 92. The method of paragraph 82, wherein R², R³, and R⁴ are each independently selected from H, OH, and C₁₋₃ alkyl.
Paragraph 93. The method of paragraph 82, wherein R², R³, and R⁴ are each H.
Paragraph 94. The method of paragraph 82, wherein:
   R³ is H;
   R⁵ is H; and
   R² and R⁴ are each independently selected from OH and C₁₋₃ alkyl.
Paragraph 95. The method of paragraph 82, wherein the compound of Formula (IV) has formula:

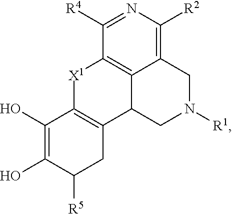

or a pharmaceutically acceptable salt thereof.
Paragraph 96. The method of paragraph 82, wherein R² and R⁴ are each independently selected from H, halo, OH, C₁₋₃ alkoxy, C₁₋₃ alkyl, and C₁₋₃ haloalkyl.
Paragraph 97. The method of paragraph 82, wherein the compound of Formula (IV) is selected from any one of the following compounds:

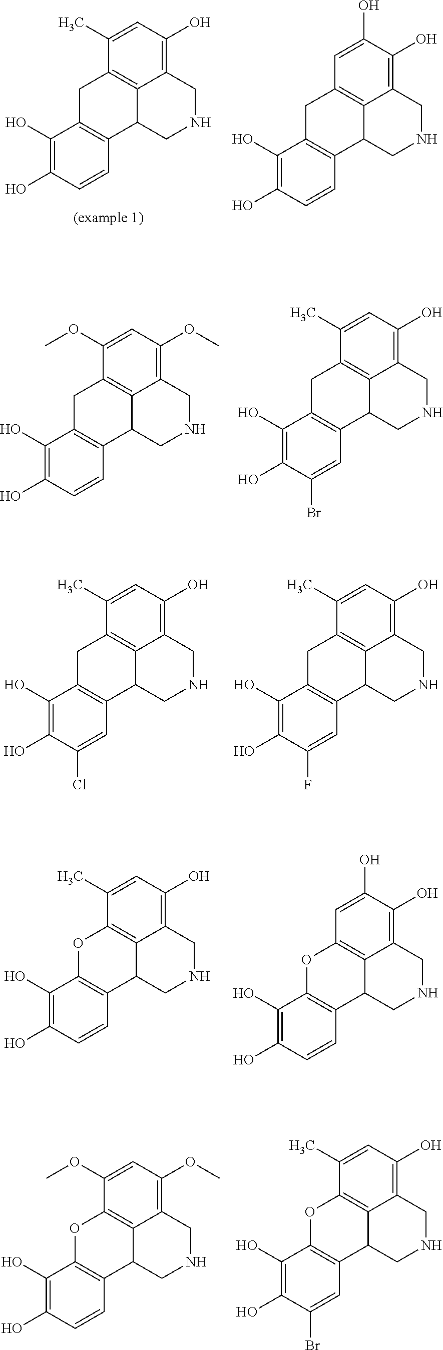

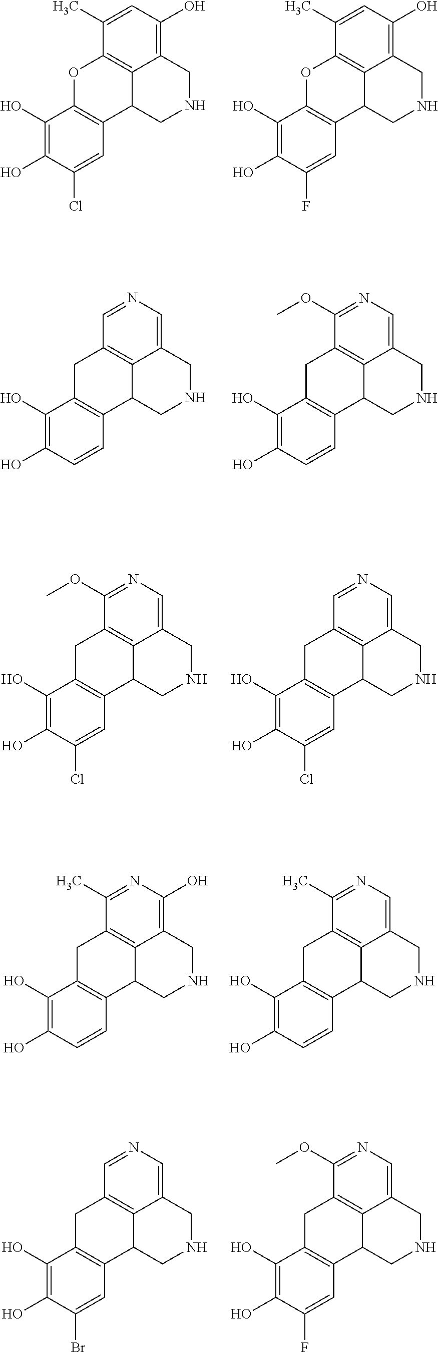

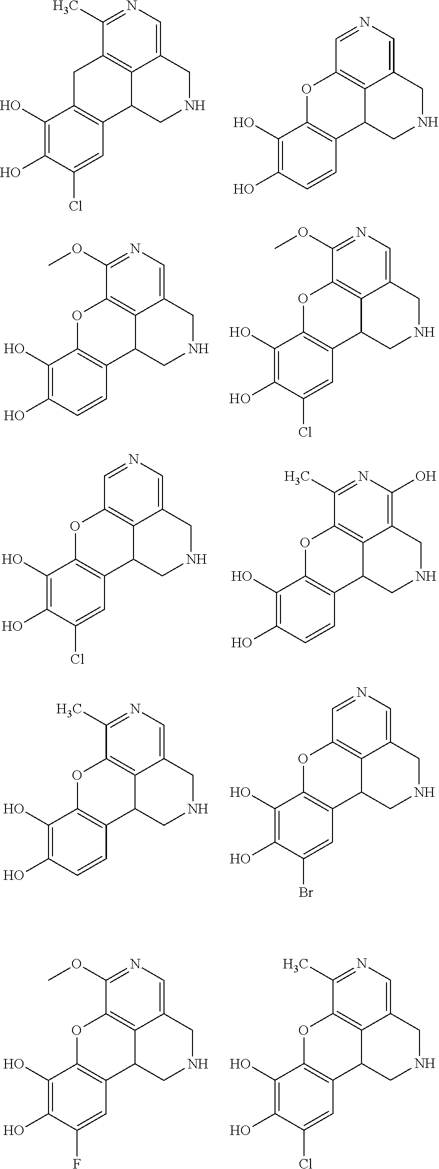

or a pharmaceutically acceptable salt thereot.
Paragraph 98. The method of any one of paragraph 1-97, wherein the administering of the compound comprises administering the compound to the subject by an ocular route.
Paragraph 99. The method of paragraph 98, wherein the administering of the compound comprises administering the compound by the ocular route selected from: intravitreal, intraocular, intracameral, subconjunctival, subtenon, intracorneal, intrastromal, trans-scleral, and suprachoroidal route.
Paragraph 100. The method of paragraph 98 or 99, wherein the administering of the compound comprises administering the compound in a pharmaceutical formulation selected from: eye-drops, eye ointment, eye emulsion.
Paragraph 101. The method of paragraph 98 or 99, wherein the administering of the compound comprises a local injection into or about cornea, choroid, retina, vitreous, uvea, orbit, eyelid, conjunctiva, or iris.
Paragraph 102. A method of inhibiting epithelial to mesenchymal transition (EMT) in a retinol pigment epithelial (RPE) cell, the method comprising contacting the cell with an effective amount of a compound as recited in any one of paragraphs 7-97, or a pharmaceutically acceptable salt thereof.
Paragraph 103. A method of inhibiting migration or proliferation of a retinol pigment epithelial (RPE) cell, the method comprising contacting the cell with an effective amount of a compound as recited in any one of paragraph 7-97, or a pharmaceutically acceptable salt thereof.
Paragraph 104. A method of inhibiting expression of a profibrotic gene in a retinol pigment epithelial (RPE) cell, the method comprising contacting the cell with an effective amount of a compound as recited in any one of paragraph 7-97, or a pharmaceutically acceptable salt thereof.
Paragraph 105. The method of paragraph 104, wherein profibrotic gene is selected from Acta2 (α-smooth muscle actin, αSMA), Ctgf(Connective tissue growth factor), Fn1 (Fibronectin), Colla1 (Collagen I), Colla2 (Collagen II), and Col3a1 (Collagen III), or any combination thereof.
Paragraph 106. A method of inhibiting extra-cellular matrix production and deposition by a retinol pigment epithelial (RPE) cell, the method comprising contacting the cell with an effective amount of a compound as recited in any one of paragraphs 7-97, or a pharmaceutically acceptable salt thereof.
Paragraph 107. A method of enhancing extra-cellular matrix degradation by a retinol pigment epithelial (RPE) cell, the method comprising contacting the cell with an effective amount of a compound as recited in any one of paragraphs 7-97, or a pharmaceutically acceptable salt thereof.
Paragraph 108. The method of any one of paragraph 102-107, wherein the contacting is carried out in vitro, in vivo, or ex vivo.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A method of treating or preventing an ocular fibrotic pathology, the method comprising administering to a subject in need thereof a therapeutically effective amount of a dopamine receptor agonist selected from dopamine receptor D1 (DRD1) agonist and dopamine receptor D5 (DRD5) agonist, or a pharmaceutically acceptable salt thereof.

2. The method of claim 1, wherein the ocular fibrotic pathology is selected from: proliferative vitreoretinopathy (PVR), diabetic retinopathy, ischemic retinopathy, age-related macular degeneration (ARMD), dry ARMD, neovascular ARMD, keratitis, pterygia, pingueculae, retinopathy of prematurity, glaucoma (including neovascular glaucoma, open-angle glaucoma, angle-closure glaucoma, secondary glaucoma, and childhood glaucoma), Stargardt's disease, sickle cell retinopathy, radiation retinopathy, optic neuropathy, retinal detachment, retinal degeneration, uveitis, dry eye disease, congenital fibrosis of the extraocular muscles (CFEOM), and corneal fibrosis.

3. The method of claim 1, wherein the ocular fibrotic pathology is selected from: opacification and fibrosis of the posterior capsule of the lens following eye surgery, fibrosis following glaucoma filtration surgery, fibrosis following a wound or trauma, conjunctival fibrosis or subconjunctival fibrosis, fibrosis of the ocular muscles, Graves disease, fibrosis following wound healing of the skin around the eye and face, fibrosis of the surface of the eye with pterygium or pingueculae, fibrosis due to choroidal neovascularization and angiogenesis, fibrosis following a corneal wound, fibrosis following corneal laser surgery, fibrosis following refractive surgery, and fibrosis following a corneal transplant.

4. The method of claim 1, wherein the dopamine receptor agonist is a compound of Formula (I): 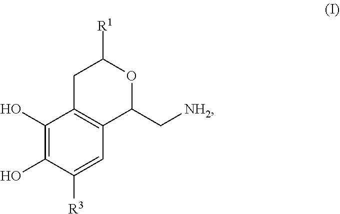
or a pharmaceutically acceptable salt thereof, wherein:
R¹ is selected from HO—C₁₋₆ alkyl, NH₂—C₁₋₆ alkyl, C₆₋₁₂ aryl ring, 5-6-membered heteroaryl ring comprising 1 to 5 heteroatoms selected from N, O, and S, and 3-10-membered heterocycloalkyl ring comprising 1 to 3 heteroatoms independently selected from N, O, and S; wherein said heteroaryl ring and heterocycloalkyl ring are each optionally substituted with 1, 2, or 3 substituents independently selected from R²;
each R² is independently selected from halo, OH, C₁₋₃ alkoxy, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, C₁₋₃ alkoxy, SH, NH₂, C₁₋₃ alkylamino, and di(C₁₋₃ alkyl)amino; and
R³ is selected from H and halo.

5. The method of claim 4, wherein the compound of Formula (I) has formula: 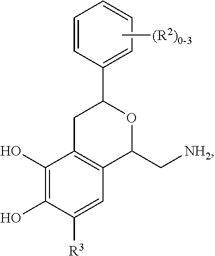
or a pharmaceutically acceptable salt thereof.

6. The method of claim 4, wherein the compound of Formula (I) is selected from: 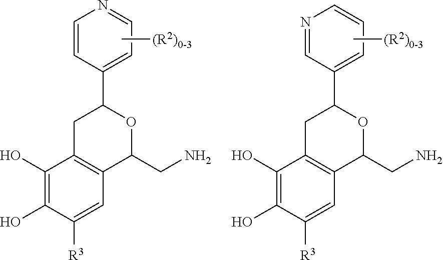
or a pharmaceutically acceptable salt thereof.

7. The method of claim 4, wherein the compound of Formula (I) is selected from: 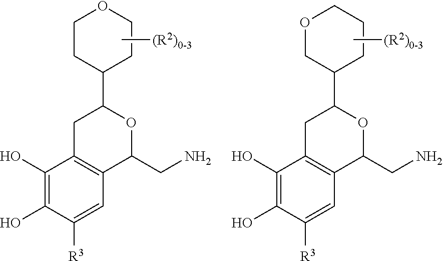
or a pharmaceutically acceptable salt thereof.

8. The method of claim 4, wherein R¹ is selected from HO—C₁₋₆ alkyl and NH₂—C₁₋₆ alkyl.

9. The method of claim 4, wherein the compound of Formula (I) is selected from any one of the following compounds: 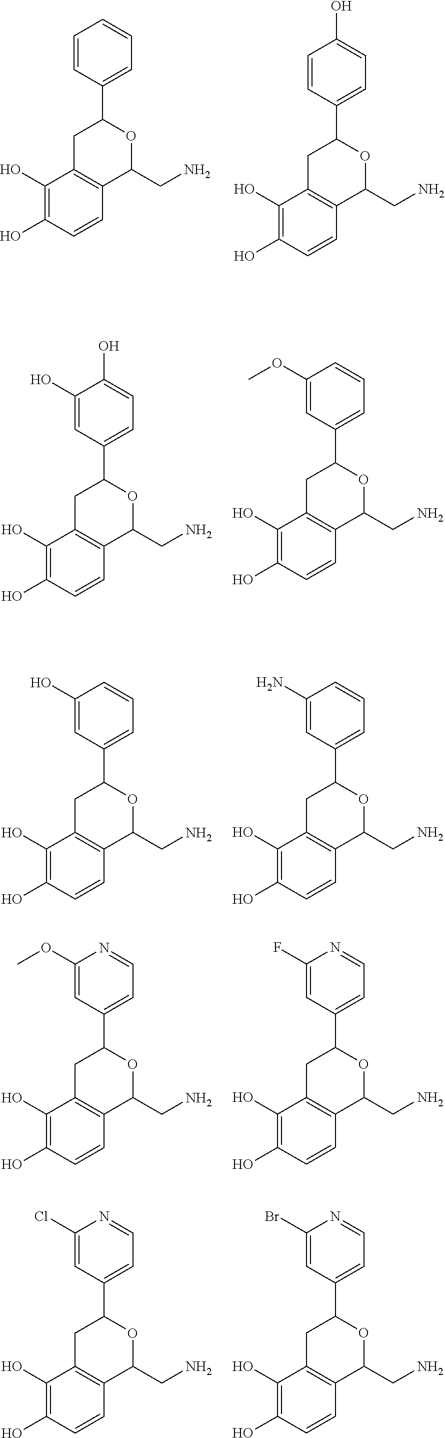 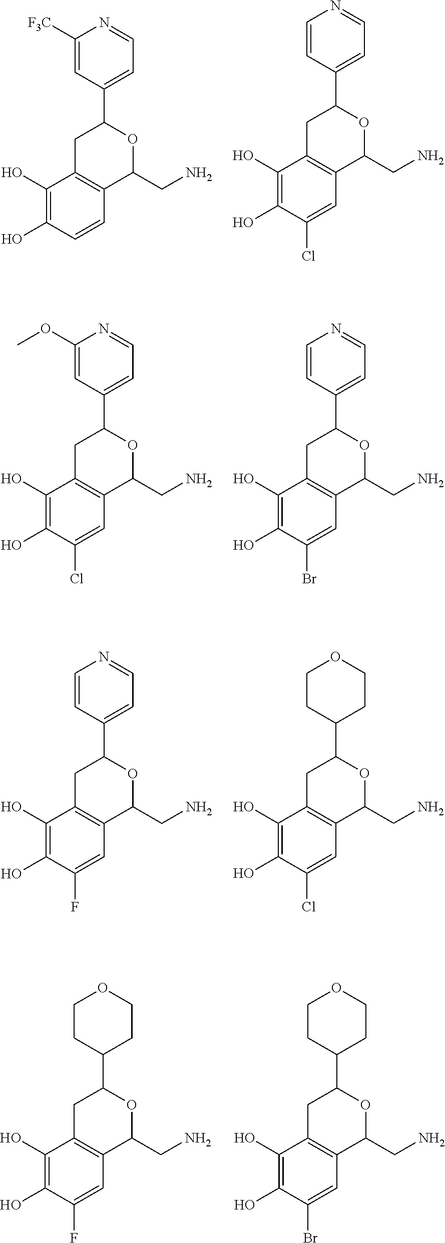
or a pharmaceutically acceptable salt thereof.

10. The method of claim 4, wherein the compound of Formula (I) is selected from any one of the following compounds: 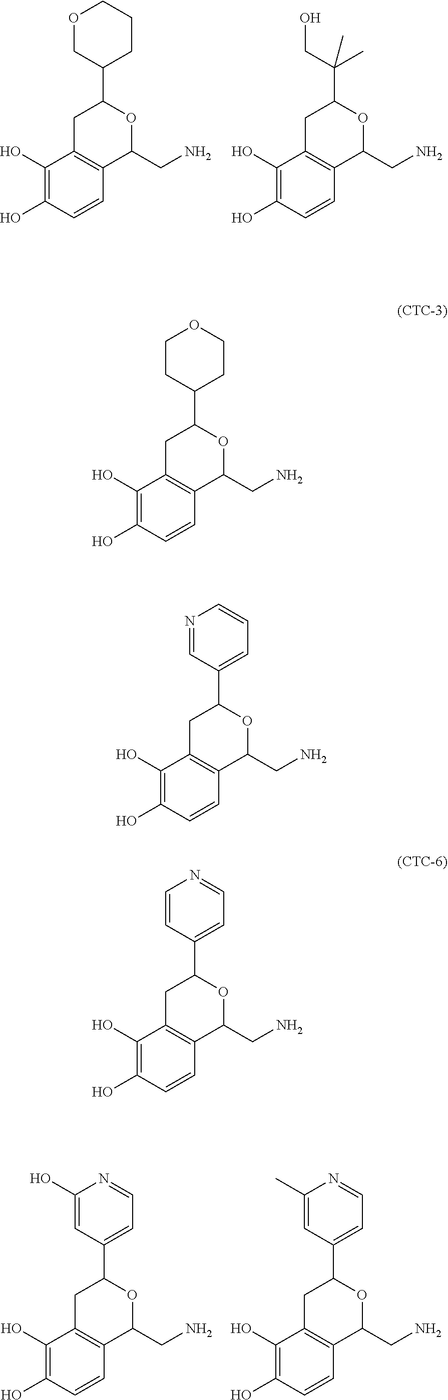
or a pharmaceutically acceptable salt thereof.

11. The method of claim 1, wherein the dopamine receptor agonist is a compound of Formula (II): 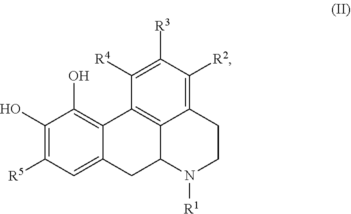
or a pharmaceutically acceptable salt thereof, wherein:
R¹ is selected from H and C₁₋₃ alkyl, wherein said C₁₋₃ alkyl is optionally substituted with OH, SH, NH₂, C₁₋₃ alkylamino, or di(C₁₋₃ alkyl)amino;
R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, SH, NH₂, C₁₋₃ alkylamino, and di(C₁₋₃ alkyl)amino; and
R⁵ is selected from H and halo.

12. The method of claim 11, wherein the compound is selected from: 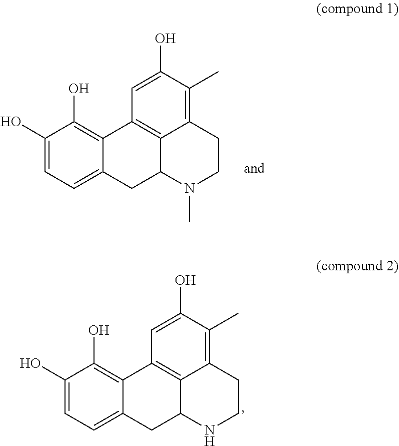 [SMILES: Cc1c(O)cc2c3c1CCN(C)C3Cc1ccc(O)c(O)c1-2 ; Cc1c(O)cc2c3c1CCNC3Cc1ccc(O)c(O)c1-2]
or a pharmaceutically acceptable salt thereof.

13. The method of claim 1, wherein the dopamine receptor agonist is a compound of Formula (III): 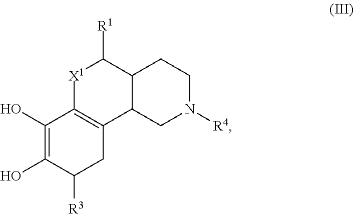
or a pharmaceutically acceptable salt thereof, wherein:
X¹ is selected from CH₂ and P;
R¹ is selected from HO—C₁₋₆ alkyl, NH₂—C₁₋₆ alkyl, C₆₋₁₂ aryl ring, 5-6-membered heteroaryl ring comprising 1 to 5 heteroatoms selected from N, O, and S, and 3-10-membered heterocycloalkyl ring comprising 1 to 3 heteroatoms independently selected from N, O, and S;
wherein said heteroaryl ring and heterocycloalkyl ring are each optionally substituted with 1, 2, or 3 substituents independently selected from R²;
each R² is independently selected from halo, OH, C₁₋₃ alkoxy, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, C₁₋₃ alkoxy, SH, NH₂, C₁₋₃ alkylamino, and di(C₁₋₃ alkyl)amino;
R³ is selected from H and halo; and
R⁴ is selected from H and C₁₋₃ alkyl, wherein said C₁₋₃ alkyl is optionally substituted with OH, SH, NH₂, C₁₋₃ alkylamino, or di(C₁₋₃ alkyl)amino.

14. The method of claim 13, wherein the compound of Formula (III) is selected from any one of the following compounds: 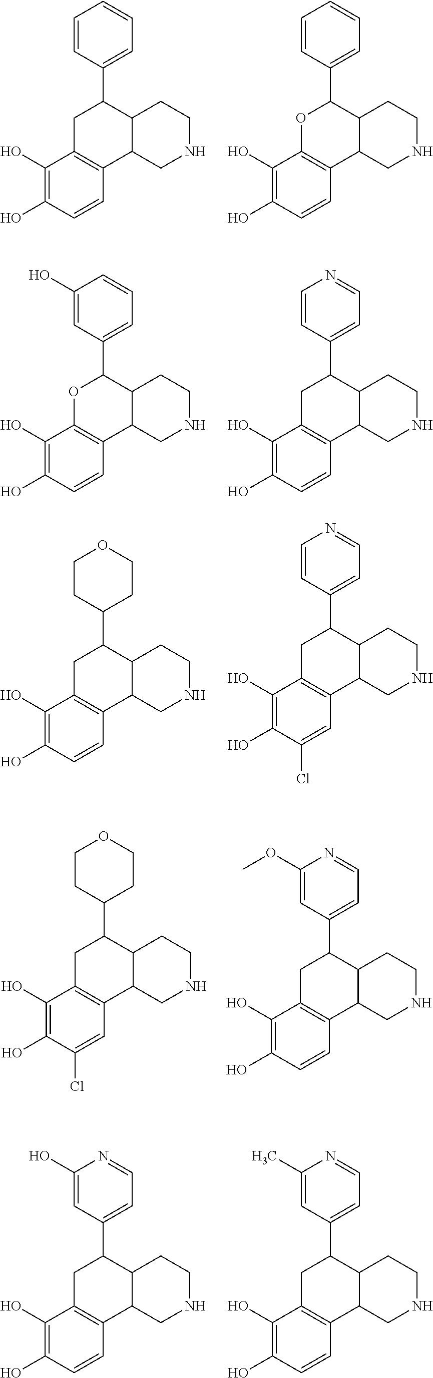 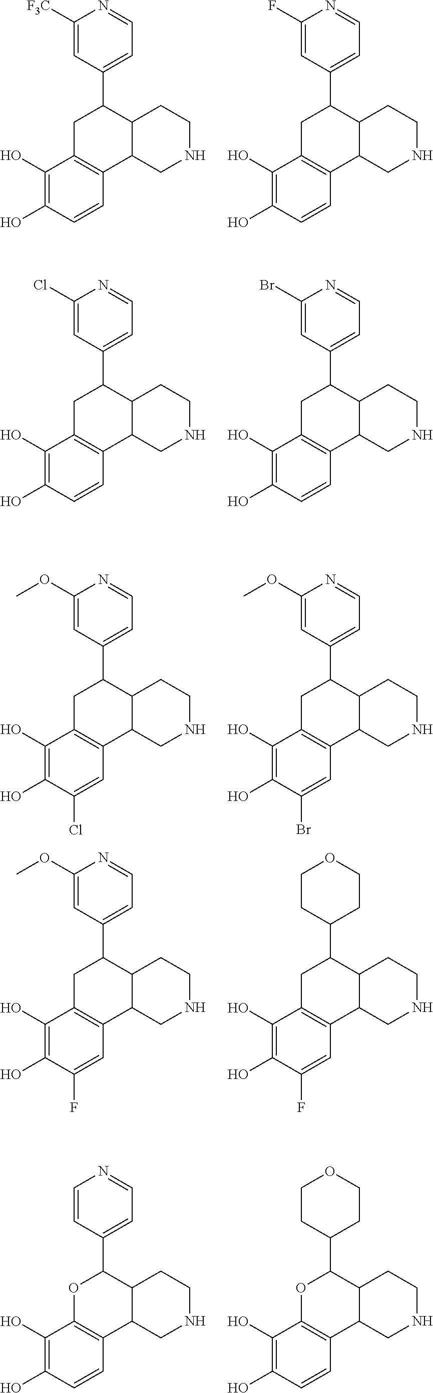 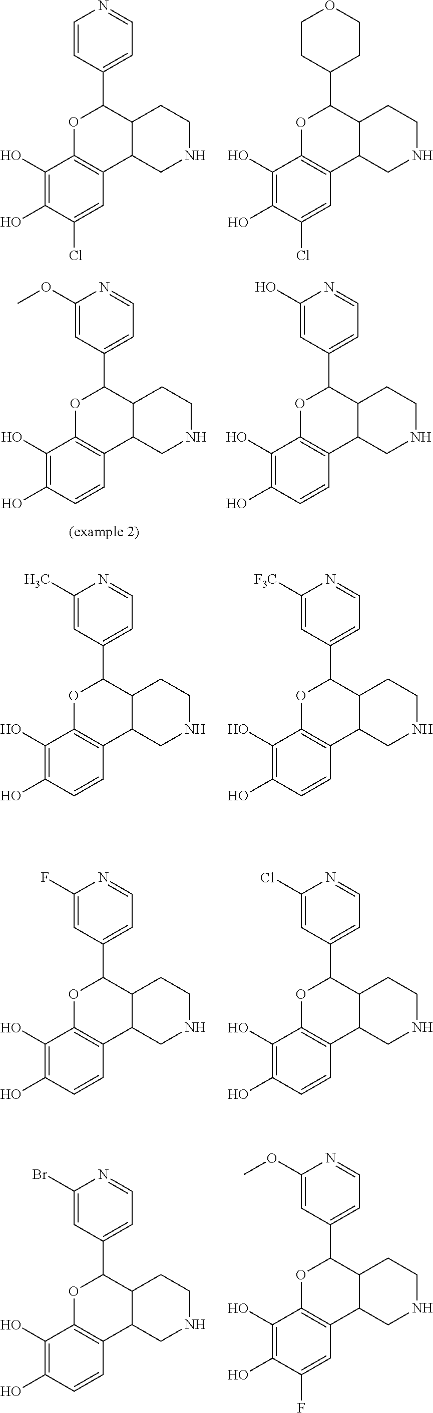
or a pharmaceutically acceptable salt thereof.

15. The method of claim 1, wherein the dopamine receptor agonist is a compound of Formula (IV): 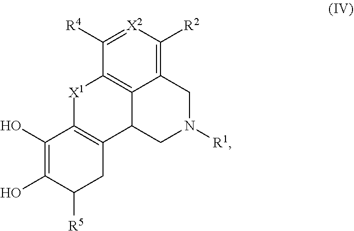
or a pharmaceutically acceptable salt thereof, wherein:
X¹ is selected from CH₂ and O;
X² is selected from CR³ and N;
R¹ is selected from H and C₁₋₃ alkyl, wherein said C₁₋₃ alkyl is optionally substituted with OH, SH, NH₂, C₁₋₃ alkylamino, or di(C₁₋₃ alkyl)amino;
R⁵ is selected from H and halo; and
R², R³, and R⁴ are each independently selected from H, OH, SH, NH₂, C₁₋₃ alkylamino, di(C₁₋₃ alkyl)amino, C₁₋₃ alkyl, and C₁₋₃ haloalkyl, wherien said C₁₋₃ alkyl is optionally substituted with OH, SH, NH₂, C₁₋₃ alkylamino, and di(C₁₋₃ alkyl)amino.

16. The method of claim 15, wherein the compound of Formula (IV) is selected from any one of the following compounds: 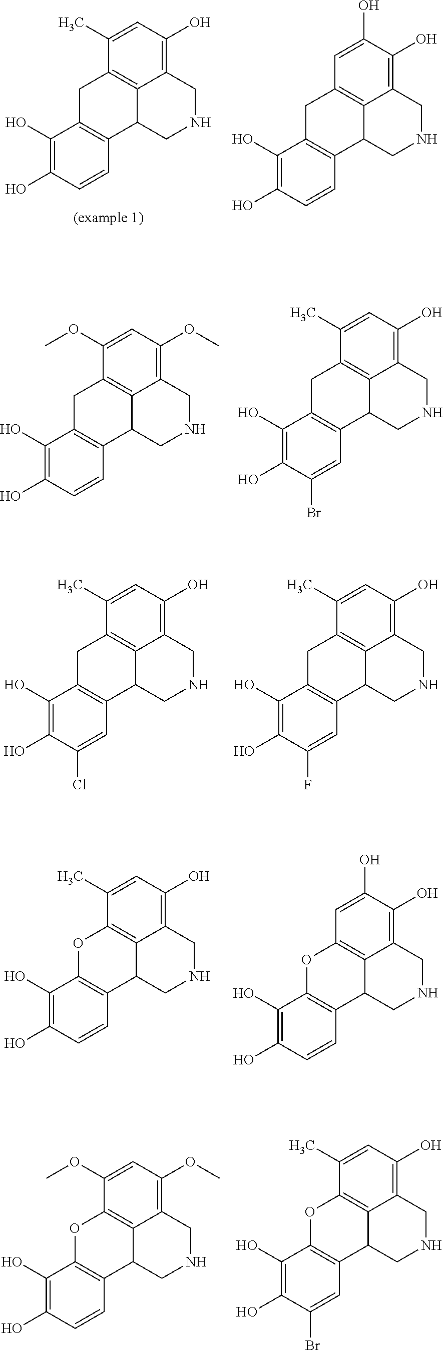 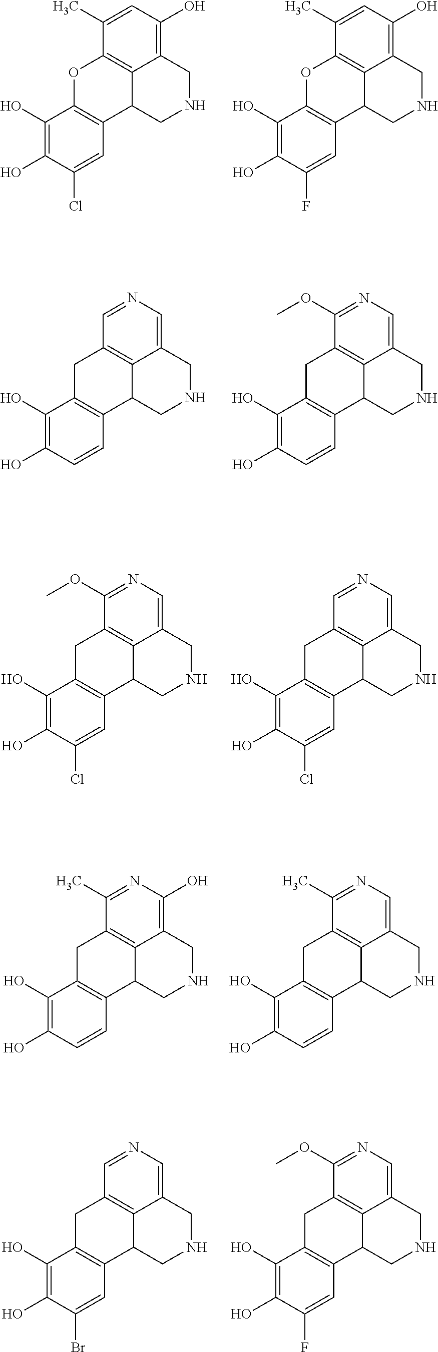 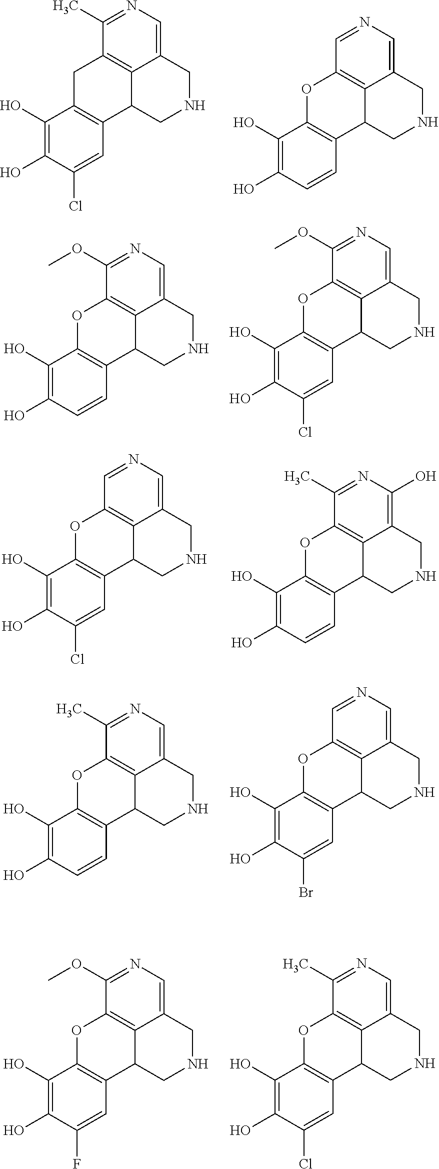
or a pharmaceutically acceptable salt thereof.

17. The method of claim 1, wherein the administering of the compound comprises administering the compound to the subject by an ocular route.

18. The method of claim 17, wherein the administering of the compound comprises administering the compound by the ocular route selected from: intravitreal, intraocular, intracameral, subconjunctival, subtenon, intracorneal, intrastromal, trans-scleral, and suprachoroidal route.

19. The method of claim 17, wherein the administering of the compound comprises administering the compound in a pharmaceutical formulation selected from: eye-drops, eye ointment, and eye emulsion.

20. The method of claim 17, wherein the administering of the compound comprises a local injection into or about cornea, choroid, retina, vitreous, uvea, orbit, eyelid, conjunctiva, or iris.
